(19) United States
(12) Patent Application Publication
(10) Pub. No.: US 2011/0256579 A1
(43) Pub. Date: Oct. 20, 2011

(54) Method for Verifying and/or Identifying Hormonally Effective Substances

(75) Inventors: Kai Ostermann, Dresden (DE); Annett Gross, Dresden (DE); Oliver Zierau, Dresden (DE); Patrick Diel, Pullheim (DE); Günter Vollmer, Pesterwitz (DE); Sylvi Lehmann, Dresden (DE); Felicitas Rataj, Dresden (DE); Gerhard Rodel, Karlsfeld (DE)

(73) Assignee: TECHNISCHE UNIVERSITAET DRESDEN, Dresden (DE)

(21) Appl. No.: 13/141,342

(22) PCT Filed: Dec. 22, 2009
(86) PCT No.: PCT/EP2009/067756

§ 371 Date: Jun. 22, 2011

(30) Foreign Application Priority Data
Dec. 22, 2008 (DE) 10 2008 064 553.2

(51) Int. Cl. C12Q 1/02; C12N 1/00

(52) U.S. Cl. 435/29; 435/255.1

(57) ABSTRACT

The invention relates to a method for verifying and/or identifying hormonally effective substances using the pheromone system of yeast, to yeast cells for carrying out the method, and to the use thereof. The method according to the invention comprises the following steps: a) adding yeast culture medium or buffer to a sample which presumably contains a hormonally effective substance, b) thereafter contacting the sample with haploid yeast cells that possess features 1 through 3: 1. the DNA sequence coded for a heterologous hormone receptor is placed under the control of a constitutive promoter, 2. the DNA sequence coded for a yeast pheromone is placed under the control of a promoter that can be regulated by the hormone receptor, 3. a gene that is coded for a receptor for the yeast pheromone is constitutively expressed, wherein the activation of the hormone receptor by the hormonally effective substance leads to the expression and secretion of the yeast pheromone, and c) the physiological and/or morphological changes of haploid yeast cells are detected.

The invention concerns a simple method for verifying and/or identifying hormonally effective substances by using the pheromone system of yeasts, yeast cells for performing the method, and the use of the method or of the yeast cells. The invention is also suitable for performing screenings in which substance mixtures and/or substance libraries are to be checked quickly and inexpensively with respect to the presence of hormonally effective substances.

In higher organisms, hormones play an important role because they allow for exchange of information between remote cells, cell groups, tissues or organs. The effect of hormones as messenger substances in this connection is not based on a direct effect on the substrates, as in case of enzymes, but on the interaction with a specific hormone receptor by means of which in the target cell a biological response is transmitted. The hormone effect is thus not caused alone by the hormone itself but also by the reactivity, for example, the receptor status of the target cell. Therefore, hormones in various target cells can cause different reactions.

Based on their chemical properties, hormones can be divided into four groups. Proteo or peptide hormones, steroid hormones, arachidonic acid derivatives (eicosanoides), and amino acid derivatives. Also, the following three groups can be distinguished:

Glandular hormones are produced in glands, neuro-secretory hormones are produced by the nerve cells. Both groups are transported subsequently through the blood stream to the target cells. This is referred to as endocrine mechanism.

In contrast, tissue hormones act locally within the tissue in which they are produced. This is referred to as paracrine mechanism.

Hormones are used in natural as well as in synthetic form in medicine and veterinary medicine for replacement of specific deficits or for obtaining of specific state, for example, as contraceptives, anti-arthritic agents, anti-diabetic agents and anabolic agents. However, hormones and hormone-like substances are also regularly abused. For example, hormones or hormone-like substances are used illegally for increasing performance (doping) in professional sports and by now also in amateur sports. Characteristic of hormones is that they are effective in concentration ranges that are usually significantly lower than those of other therapeutics; this makes their detection difficult and very sensitive sensory means are required.

Anabolic substances, in particular anabolic steroids, are used frequently for doping. The desired effect of the anabolic substances is based on improved fat burning and simultaneous muscle generation. They ensure a positive nitrogen balance and thus also muscle-anabolic metabolic state. In addition to natural androgens, increasingly synthetic steroids that exhibit an action similar to the male sex hormone testosterone are used for doping.

Because of the widespread use of hormones in the medical field as well as for illegal performance improvement the environmental loading by hormonally active substances has increased greatly. Administered hormones are partially excreted again together with the urine and thus reach bodies of water because they are not filtered by conventional water treatment plants from the wastewater. Since hormones and hormonally active substances bind with high affinity to the corresponding receptors and already in smallest quantities develop their effectiveness, the loading of bodies of water is a great problem for humans and animals.

Aside from synthetic hormones that reach the environment, also natural substances can have a hormonal action. Many plant-based substances have a hormonal action and are therefore also called phyto-hormones. A positive effect is often attributed to these substances so that they are also used therapeutically, for example, in case of menopausal disorders.

The classic analytical method for detecting steroid hormones is gas chromatography, followed by mass spectrometry (GC/MS). A disadvantage of this method resides in that in principle only known substances can be detected. This is, for example, problematic when for the purpose of doping synthetic substances are used that are not yet known to the doping investigators. Such substances can hardly be detected with conventional methods.

For identifying new hormonally active substances existing in the environment that are not hormones but can develop a hormonal action in the human or animal organism (so-called xenohormones that also include the aforementioned phyto-hormones), such methods are also suitable only to a limited extent.

A significant improvement in regard to this problem are biological testing methods that recognize substances by means of binding to the corresponding receptors. In this connection, very complex and cost intensive mammalian cell systems as well as significantly simpler and cheaper yeast systems exist. Two frequently employed yeast systems are the estrogen receptor-α and the androgen receptor yeast assay of Sumpter and co-workers (Routledge and Sumpter, 1996; Sohoni and Sumpter, 1998). Here, the receptor binding causes an increase of an enzyme activity that then leads to a color change of the medium or bio luminescence. The prior solutions according to the aforementioned yeast assays have the disadvantage that chemical reactions, for example, the color reactions in the β-galactosidase or phytase-based method, or physical parameters, such as fluorescence of proteins, are required for detecting. Moreover, they provide no possibility of an intrinsic signal amplification.

Object of the invention is to provide a simple method for detecting and/or for identifying hormonally active substances that can be realized even with very simple technical means and with which, where appropriate, the detection limit for the hormonally active substances to be detected is lowered. Object of the invention is furthermore to provide a method for screening substance libraries and substance mixtures with which the presence of hormonally active substances can be'evaluated quickly and inexpensively.

The object is solved by a method for detecting and/or identifying hormonally active substances by utilizing the pheromone systems of yeasts. The method according to the invention comprises the following steps:
a) to a sample that comprises presumably a hormonally active substance a yeast culturing medium or buffer is added,
b) the sample to which the yeast culturing medium or buffer has been added is contacted with haploid yeast cells that comprises the following features 1 to 3, wherein either all features 1 to 3 are realized by a single type of haploid yeast cells or the features 1 and 2 are realized by a first type of haploid yeast cells and the feature 3 by a second type of haploid yeast cells:
   1. the DNA sequence that codes for a heterologous hormone receptor is subjected to the control of a constitutive promoter and is functionally expressed;
   2. the DNA sequence coding for a yeast pheromone is subjected to the control of a promoter that can be regulated by the heterologous hormone receptor,
   3. a gene that codes for a receptor for the yeast pheromone is constitutively and functionally expressed,
wherein the activation of the heterologous hormone receptor by the hormonally active substance causes the expression of the DNA sequence coding for the yeast pheromone and the secretion of the yeast pheromone from the haploid yeast cell; and
c) the physiological and/or morphological changes of the haploid yeast cells are detected.

In the presence of a hormonally active substance, the hormone receptor that is expressed by the haploid yeast cells is activated. This activation causes the expression and secretion of the yeast pheromone that is under the control of a promoter that can be regulated by the hormone receptor.

Haploid yeast cells that have a receptor for the expressed and secreted yeast pheromone react to binding of the yeast pheromone with physiological and/or morphological changes that are subsequently detected. The invention utilizes in this connection the pheromone system of yeasts that is used during yeast mating. The method according to the invention therefore provides the possibility to detect and/or identify a hormonally active substance by means of the yeast pheromone system, preferably by simple methods, particularly preferred light-microscopic methods.

An aspect of the invention is therefore also haploid yeast cells that are suitable for performing the method, having the following features:
1. the DNA sequence that codes for a heterologous hormone receptor is subjected to the control of a constitutive promoter and is functionally expressed;
2. the DNA sequence coding for a yeast pheromone is subjected to the control of a promoter that can be regulated by the hormone receptor;
   wherein the activation of the heterologous hormone receptor by the hormonally active substance causes the expression of the DNA sequence coding for the yeast pheromone and the secretion of the yeast pheromone.

For performing the method according to the invention moreover the presence of haploid yeast cells is required that have the following third feature:
3. a gene that codes for a receptor for the yeast pheromone is constitutively and functionally expressed.

In one embodiment of the invention the haploid yeast cells that express a heterologous hormone receptor recombinantly (feature 1) and in which a DNA sequence coding for a yeast pheromone is subjected to the control of a promoter that is regulated by a heterologous hormone receptor (feature 2), are not those yeast cells that have a, receptor for the yeast pheromone (feature 3). The invention thus comprises haploid yeast cells of a first type that comprise the features 1 and 2 and haploid yeast cells of a second type that comprise the feature 3. Preferably, these two cell types have different mating types. In this embodiment, the signal that is triggered by the hormonally active substance is advantageously amplified intrinsically by the yeast pheromone system and the sensitivity of the method is increased.

In another embodiment of the invention, in the method according to the invention a uniform type of the haploid yeast cells is employed that comprise a heterologous hormone receptor, a gene that is subjected to the control of the heterologous hormone receptor for a yeast pheromone, and a receptor for this yeast pheromone, i.e., all features 1 to 3 are encompassed. This enables advantageously a simpler manipulation of the employed yeast cells.

The hormonally active substances are preferably present in complex mixtures such as urine samples, blood samples, plasma samples, serum samples, plant extracts or tissue extracts. However, synthetic or natural substance libraries may also be concerned in which substances are present in pure form or as combination preparations of individual substances. For contacting with the yeast cells, to the samples of these substances or substance mixtures yeast culturing medium or buffer is added. Preferably, the proportion of the yeast culturing medium or of the buffer is more than 50% up to 99.9%, preferably more than 20% up to 99.9%, of the mixture. The samples can also be directly mixed in the desired ratio with the haploid yeast cells that are contained in yeast culturing medium. The proportion of the sample relative of the total volume when contacting the yeast cells with the sample is preferably 0.01% up to 50%, preferably 0.01% to 20%.

Yeast culturing media according to the invention are yeast culturing media known to a person skilled in the art such as YPD (yeast peptone dextrose), YPDA (yeast peptone dextrose adenine), nutrient deficient media such as SD (synthetic defined), MM (minimal medium), SMM (supplemented medium) etc. Nutrient deficient media contain preferably select amino acids and other essential substances in order to enable culturing of haploid yeast cells with certain auxotrophies or of haploid yeast cells that have a specific growth marker. The composition of such media is known to a person of skill in the art and can be found in relevant literature, for example, Kaiser et al. (1994), or on Internet websites of various laboratories working with yeast.

The buffers according to the invention are all buffers that do not impair the survival and responsiveness of the haploid cells. They include, for example, PBS (phosphate-buffered saline), TBS (tris-buffered saline), and can be found in relevant literature, such as Kaiser et al. (1994).

By binding of the yeast pheromone to the pheromone receptor, a complex mating program (“mating response pathway”) is activated in the responsive yeast cells (Leberer et al., 1997). Mating-specific genes are induced and the cell cycle is arrested. Subsequently, complex morphological changes occur such as a directed cell wall growth (mating projection) of the cells toward the source of the yeast pheromone, in general toward the mating partner (Jackson et al., 1991; Segall, 1993). This “extension” of the yeast cells is referred to as “shmoo” (Mackay and Manney, 1974).

Within 1-2 hours after activation of the pheromone receptor by binding of the yeast pheromone the morphological changes are visible very well. In addition to the directed growth toward the mating partner that is induced by the corresponding yeast pheromone, one observes in cell populations of different mating types the fusion of the cells and the formation of characteristically shaped zygotes (Tkacz and MacKay, 1979). The entire process of mating up to the diploid zygote can be effectively and simply followed by means of a light microscope. First, on the cell surface of the cells of different mating type agglutinins are exposed so that binding of the cells to each other or clumping occurs. The cells adhere; fusion of the cell walls, of the cell membranes, cytogamy and karyogamy occur (Marsh and Rose, 1997).

These morphological changes caused by the action of the yeast pheromone can be observed advantageously by a light microscope. For this purpose a magnification by at least 100 times, preferably 200 times up to 1,000 times, is suitable.

The detection is also measurable by the turbidity of the solution in which the haploid yeast cells according to the invention are contained and can be detected by means of a nephelometer. These measurements can advantageously be performed with minimal technical expenditure.

A further morphological change that goes hand-in-hand with yeast mating and that can be used for detection of the androgen action is the cell cycle arrest that is caused by the pheromones secreted during mating. The pheromone of one mating type causes in cells of the other mating type blocking of mitosis as a preparation to mating and the subsequent meiosis.

This effect can be utilized in order to detect the hormonal action of a substance in the method according to the invention. When an aliquot of haploid yeast cells according to the invention that comprise at least the features 1 and 2 is incubated with a hormonally active substance and then applied spotwise onto an agar plate that has been inoculated across the surface with strongly diluted haploid yeast cells that comprise at least the feature 3, then the latter cells, as a result of the secreted yeast pheromone of the yeast cells incubated with the substance to be tested, stop their growth. In other areas of the agar plate, the yeast cells can however grow without inhibition. Therefore, in areas where the yeast cells incubated with the substance to be tested have been applied spotwise, a zone without yeast cells (zone of inhibition) visible to the naked eye is formed.

As an alternative, or in addition, to the detection of the morphological changes in the haploid yeast cells physiological changes are detected. For this purpose, in the haploid yeast cells that exhibit feature 3, a specific gene that, for example, codes for a marker protein, e.g. GFP (green-fluorescent protein) is subjected to the control of a promoter is that is regulated by the yeast pheromone that is secreted by the haploid yeast cells with the features 1 and 2. This means that the haploid yeast cells are gene-technologically changed such that they react to activation of the yeast pheromone receptor by physiological changes that can be physically or chemically detected. When the yeast pheromone that has been expressed and secreted by binding of the hormonally active substance to the heterologous hormone receptor reaches the surrounding cells that have a receptor for the yeast pheromone, the expression of the specific gene and the production of the marker protein or target protein by the yeast pheromone-regulated promoter are caused in these cells as a physiological change.

The marker protein is for example β-galactosidase, EGFP or phytase. By the expression of the specific gene that is yeast pheromone-controlled, a detectable signal, for example, color reactions or fluorescence is generated. In this way, advantageously the detection according to the invention of the hormone is also enabled and facilitated by detection of measuring parameters other than the morphological changes of the yeast cells, for so example, by fluorescence measurement or by enzymatic color reactions.

Marker proteins in the context of the invention are therefore all proteins whose presence or activity causes a physically and/or chemically measurable change. This physically and/or chemically measurable change is detected by a suitable detection system in a simple and/or fast way. Preferably, those marker proteins are used that can be detected without impairing the integrity or vitality of the cells, for example, enzymes that in the presence of a substrate catalyze a color reaction such as β-galactosidase or phytase. Furthermore, luciferases that emit light in the presence of a suitable substrate are preferred as marker proteins. Especially preferred as marker proteins are proteins that, when excited, fluoresce with light of a certain wavelength. Moreover, the invention comprises proteases that decompose fluorescent proteins as marker proteins. When simultaneously the haploid yeast cell expresses constitutively a fluorescent protein, the decrease of fluorescence of the cells is measurable as a result of the detection of the hormone. Preferred are proteases that do not attack, aside from the fluorescent protein, any other targets in the cell in order not to impair the vitality of the cell. Especially preferred is the TEV protease. The corresponding fluorescent proteins, if appropriate, must be changed by recombinant DNA techniques such that they contain the recognition sequence for the corresponding protease and therefore can be decomposed.

The marker protein is preferably a fluorescent protein wherein the expression of the corresponding marker protein after secretion of the yeast pheromone by the haploid yeast cells with the features 1 and 2 varies; this leads to an increase or decrease of fluorescence of the haploid yeast cells with the feature 3. Preferably used are the fluorescent proteins GFP, YFP, CFP, BFP, RFP, DsRed, PhiYFP, JRed, emGFP (“emerald green”), Azami Green, Zs-Green, or AmCyan 1. Preferred are proteins that have been changed such that they fluoresce particularly strongly, such as eGFP, GFPuv, eYFP, TagCFP, TagGFP, TagYFP, TagRFP and TagFP365. Furthermore, preferred are those fluorescent proteins whose amino acid sequence is changed in that they fluoresce after an aging time that is as short as possible. Preferred are in this respect: TurboGFP, TurboYFP, TurboRFP, TurboFP602, TurboFP635, and dsRed-Express.

Especially preferred is that the specific gene that is regulated by the yeast pheromone codes for a protein as a marker protein that fluoresces green (e.g. GFP, GFPuv), yellow (e.g. YFP), blue (e.g. BFP), cyan (e.g. CFP), or red (e.g. dsRed).

By means of a suitable detection system for the marker protein the formation of the marker protein is detected sensor-technologically. Preferably the formation of the marker protein is detected by a fluorimeter, a spectrometer, a microscope, a plate reader, or by flow cytometry.

The hormone receptors according to the invention are heterologous hormone receptors because they are not present authentically in the employed haploid yeast cells but originate from other organisms. They are expressed by the haploid yeast cells in that a DNA sequence that codes for the heterologous hormone receptor is introduced into the haploid yeast cells. The heterologous hormone receptors are preferably intracellular, preferably cytoplasmic or nuclear, hormone receptors. Low molecular weight hormones, for example, steroid hormones that can diffuse through cell and nucleus membranes bind in general to such intracellular receptors. The receptors are often located nuclear, i.e., within the nucleus of the cell. Such nuclear receptors are thus also referred to as nuclear receptors or nucleus receptors. They are comprised generally of a hormone-binding domain and a DNA-binding domain. By DNA binding they participate in the regulation of certain cell genes. These nuclear receptors are usually proteins with a zinc finger domain that is responsible for DNA binding. Examples of such nuclear receptors are the receptors for the thyroid hormone (triiodo thyronine), for the steroid hormones, for vitamin D3, for retinoic acid, and bile acids.

These intracellular or nuclear hormone receptors enable advantageously the detection of hormones without having to make sure that the employed haploid yeast cells indeed have a suitable second messenger system for transmitting the hormone signals to the cell nucleus. Intracellular and nuclear hormone receptors bind advantageously directly to the DNA in the nucleus and regulate in this way the gene expression.

Hormone receptors are highly specific and moreover have high affinity because hormones in the body usually exist in only minimal quantities. In this way, they advantageously enable the highly sensitive detection of minimal quantities of hormonally acting substances. Preferred hormone receptors are listed in Table 1. Preferably, receptors for steroid hormones are used; it is especially preferred to select the hormone receptors according to the invention from estrogen receptors and androgen receptors.

TABLE 1
Preferred hormone receptors according to the invention

| Sub | | | | GeneDatabase |
|---|---|---|---|---|
| families/ | | | | Accession |
| Groups | Gene | Common name | Alternative names | Number |
| 1A | NR1A1 | TRa, c-erbA-1, THRA | Thyroid hormone | M24748 |
| | | | receptor-a | |
| | NR1A2 | TRb, c-erbA-2, THRB | Thyroid hormone | X04707 |
| | | | receptor-b | |
| 1B | NR1B1 | RARa | Retinoic Acid receptor-a | X06538 |
| | NR1B2 | RARb, HAP | Retinoic Acid receptor-b | Y00291 |
| | NR1B3 | RARg, RARD | Retinoic Acid receptor-g | M57707 |
| | NR1B4 | RAR | Retinoic Acid receptor | AF378827 |
| 1C | NR1C1 | PPARa | Peroxisome Proliferator | L02932 |
| | | | activated receptor-a | |
| | NR1C2 | PPARb, NUC1, | Peroxisome Proliferator | L07592 |
| | | PPARd, FAAR | activated receptor-b | |
| | NR1C3 | PPARg | Peroxisome Proliferator | L40904 |
| | | | activated receptor-g | |
| 1D | NR1D1 | REVERBa, EAR1, | | M24898 |
| | | EAR1A | | |
| | NR1D2 | REVERBb, EAR1b, | | L31785 |
| | | BD73, RVR, HZF2 | | |
| | NR1D3 | E75 | | X51548 |
| 1E | NR1E1 | E78, DR-78 | | U01087 |
| 1F | NR1F1 | RORa, RZRa | retinoid-related orphan | U04897 |
| | | | receptor-a | |
| | NR1F2 | RORb, RZRb | retinoid-related orphan | Y08639 |
| | | | receptor-b | |
| | NR1F3 | RORg, TOR | retinoid-related orphan | U16997 |
| | | | receptor-g | |
| | NR1F4 | HR3, DHR3, MHR3, | | M90806 |
| | | GHR 3 | | |
| | | CHR3, CHR3 | | U13075 |
| 1G | NR1G1 | CNR14 | | U13074 |
| 1H | NR1H1 | ECR | | M74078 |
| | NR1H2 | UR, OR-1, NER1, | Liver X receptor-b | U07132 |
| | | RIP15, LXRb | | |
| | NR1H3 | RLD1, LXR, LXRa | Liver X receptor-a | U22662 |
| | NR1H4 | FXR, RIP14, HRR1 | Farnesoid X receptor | U09416 |
| | NR1H5 | FXRB | Farnesoid X receptor-b | AY094586 |
| 1I | NR1I1 | VDR | Vitamin-D receptor | J03258 |
| | NR1I2 | ONR1, PXR, SXR, | Pregnan-X receptor | X75163 |
| | | BXR | | |
| | NR1I3 | MB67, CAR1, CARa | | Z30425 |
| | NR1I4 | CAR2, CARb | | AF009327 |
| 1J | NR1J1 | DHR96 | | U36792 |
| 1K | NR1K1 | NHR1 | | U19360 |
| 2A | NR2A1 | HNF4 | Hepatic nuclear factor 4 | X76930 |
| | NR2A2 | HNF4G | Hepatic nuclear factor 4G | Z49826 |
| | NR2A3 | HNF4B | Hepatic nuclear factor 4B | Z49827 |
| | NR2A4 | DHNF4, HNF4D | | U70874 |
| 2B | NR2B1 | RXRA | Retinoic X receptor-a | X52773 |
| | NR2B2 | RXRB, H-2RIIBP, | Retinoic X receptor-b | M84820 |
| | | RCoR-1 | | |
| | NR2B3 | RXRG | Retinoic X receptor-g | X66225 |
| | NR2B4 | USP, Ultraspiracle, | | X52591 |
| | | 2C1, CF1, RXR1, RXR2 | | |
| 2C | NR2C1 | TR2, TR2-11 | | M29960 |
| | NR2C2 | TR4, TAK1 | | L27586 |
| | NR2C3 | TR2-4 | | AF378828 |
| 2D | NR2D1 | DHR78 | | U36791 |
| 2E | NR2E1 | TLL, TLX, XTLL | | S72373 |
| | NR2E2 | TLL, Tailless | | M34639 |
| | NR2E3 | PNR | | AF121129 |
| | NR2E4 | dissatisfaction | | O96680 |
| | NR2E5 | fax-1 | | Q9U410 |
| 2F | NR2F1 | COUP-TFI, | Chicken ovalbumin | X12795 |
| | | COUPTFA, EAR3, | upstream promoter- | |
| | | SVP44 | transcription factor-I | |
| | NR2F2 | COUP-TFII, | Chicken ovalbumin | M64497 |
| | | COUPTFB, ARP!, | upstream promoter- | |
| | | SVP40 | transcription factor-II | |
| | NR2F3 | SVP, COUP-TF | | M28863 |
| | NR2F4 | COUP-TFIII, | | X63092 |
| | | COUPTFG | | |
| | NR2F5 | SVP46 | | X70300 |
| | NR2F6 | EAR2 | | X12794 |
| | NR2F7 | AmNR7 | | AF323687 |
| 2G | NR2G1 | HNF, RXR | | AJ517420 |
| 2H | NR2H1 | AmNR4, AmNR8 | | AF323683 |
| 3A | NR3A1 | ERa | Estrogen receptor-a | X03635 |
| | NR3A2 | ERb | Estrogen receptor-b | U57439 |
| 3B | NR3B1 | ERR1, ERRa | Estrogen related | X51416 |
| | | | receptor-a | |
| | NR3B2 | ERR2, ERRb | Estrogen related | X51417 |
| | | | receptor-b | |
| | NR3B3 | ERR3, ERRg | Estrogen related | AF094318 |
| | | | receptor-g | |
| | NR3B4 | Drosophila ERR | | AE003556 |
| 3C | NR3C1 | GR | Glucocorticoid receptor | X03225 |
| | NR3C2 | MR | Mineralocorticoid receptor | M16801 |
| | NR3C3 | PR | Progesteron receptor | M15716 |
| | NR3C4 | AR | Androgen receptor | M20132 |
| 4A | NR4A1 | NGFIB, TR3, N10, | | L13740 |
| | | NUR77, NAK1 | | |
| | NR4A2 | NURR1, NOT, RNR1, | | X75918 |
| | | HZF-3, TINOR | | |
| | NR4A3 | NOR1, MINOR | | D38530 |
| | NR4A4 | DHR38, NGFIB | | U36762 |
| | | CNR8, C48D5 | | U13076 |
| 5A | NR5A1 | SF1, ELP, FTZ-F1, | | D88155 |
| | | AD4BP | | |
| | NR5A2 | LRH1, xFF1rA, | | U93553 |
| | | xFF1rB, FFLR, PHR, | | |
| | | FTF | | |
| | NR5A3 | FTZ-F1 | | M63711 |
| | NR5A4 | FF1b | | Q9IAI9 |
| 5B | NR5B1 | DHR39, FTZF1B | | L06423 |
| 6A | NR6A1 | GCNF1, RTR | | U14666 |
| | NR6A2 | HR4, THR4, GRF | | AL035245 |
| 0A | NR0A1 | KNI, Knirps | | X13331 |
| | NR0A2 | KNRL, Knirps related | | X14153 |
| | NR0A3 | EGON, Embryonic | | X16631 |
| | | gonad, EAGLE | | |
| | NR0A4 | ODR7 | | U16708 |
| | NR0A5 | Trithorax | | M31617 |
| 0B | NR0B1 | DAX1, AHCH | | S74720 |
| | NR0B2 | SHP | | L76571 |

An especially preferred hormone receptor of the invention is the human androgen receptor (hAR) to which androgenically active hormones such as testosterone or its metabolite dihydro testosterone can bind. It belongs to the family of nuclear receptors. There exist two isoforms of the receptor, isoform 1 (87 kDa) and isoform 2 (110 kDa) (Wilson and McPhaul, 1994) which are coded by the hAR gene. The isoforms differ from each other in that the isoform 1 lacks 23 amino acids at the N-terminus. Structurally, the androgen receptor is comprised of four functional domains. At the N-terminus it has an N-terminal domain by means of which primarily the activation of the receptor is regulated. In this connection, the activation by androgenically active hormones such as testosterone or dihydro testosterone (Roy et al., 1999) as well as the constitutive activation without ligand is controlled (Jenster et al., 1995). Moreover, this domain plays a role in the dimerization of the receptor (Langley et al., 1995; Doesburg et al., 1997). The N-terminal domain is followed by the strongly preserved DNA binding domain with which the receptor binds to the DNA. Between DNA binding domain and ligand binding domain there is the so-called hinge region. This is a flexible hardly preserved section of the receptor. This region coordinates the localization of the receptor in the cell nucleus and contains a part of the nuclear signal sequence. At the C-terminus of the receptor protein there is the ligand binding domain. This section has moreover an activation function that is important for agonists. In this region also co-activators can bind (Dubbink et al., 2004).

The function of the androgen receptor can be differentiated into a genomic and a non-genomic function. The main function of the receptor is the genomic one. The binding of the ligand effects inter alia the dimerization of the receptor protein and the transport of the receptor ligand complex into the nucleus. Here the construct binds to a specific DNA sequence, the hormone-responsive element. Accordingly, the androgen receptor is a transcription factor that controls the expression of genes (Mooradian et al., 1987). Examples of the non-genomic function are the activation of the MAPK signal cascade and the control of the intracellular calcium concentration.

Hormone receptors in the meaning of the invention are not only the natural proteins but also derivatives thereof, for example, chimeric proteins that by fusion of the hormone receptor with other proteins or protein domains or by linking of different domains of different hormone receptors create new proteins, for example, by molecular biological techniques.

The use of hormone receptors enables advantageously also the detection of unknown hormonally active substances, i.e. substances that are not known, or of known substances whose hormonal activity is not known. The method is suitable therefore for the detection of hormonally active substances in samples as well as for a simple and inexpensive screening of substances for evaluating their hormonal activity.

Hormonally active substances according to the invention include hormones. Hormones are biochemically non-uniform substances that within an organism transmit signals and messages to cells, cell groups, tissue or organs that are remote from the site of generation of the hormones and in this way have a certain physiological effect on their function. The hormone receptors according to the invention bind primarily to steroid hormones but also to other low molecular weight hormones that are sufficiently lipophilic to be able to diffuse through the cell membrane or optionally the nucleus membrane. Hormones according to the invention are non-proteo hormones, for example, steroids (steroid hormones), biogenic amines (L-thyroxine, melatonine, catecholamine) and fatty acid derivatives (prostaglandines and other eicosanoides).

Hormones that can be detected by the method according to the invention are, for example, steroid hormones such as estrogens (for example, estradiol, estron, and estriol), androgens (for example, androsterone, testosterone, dihydro testosterone), gestagens (for example, progesterone), glucocorticoids (for example, cortisol, corticosterone, cortisone), or mineralocorticoids (for example, aldosterone), and also thyrosine derivatives such as adrenaline, melatonine, levothyroxine (tetraiodo thyronine) and liothyronine (triiodo thyronine) or vitamins such as cholecalciferol (for short calciol or also vitamin D3) or retinol (vitamin A).

Hormones in the meaning of the invention are in this connection the natural hormones as well as the chemically synthesized compounds that are structurally identical to natural to hormones. Preferred hormones of the invention are vertebrate hormones, in particular mammalian hormones.

In addition to natural hormones, hormonally active substances in the meaning of the invention are also hormone-like substances that as a result of their structure are capable of exerting the same or similar actions as natural hormones. They include substances that are referred to as xenohormones, for example, phytoestrogens. These are in general non-steroid substances that nonetheless can activate the hormone receptor. The hormonally active substances include also synthetic structural analogues of natural hormones that can bind to the same receptors as natural hormones. They include, for example, so-called designer steroids such as norbolethone, nandrolone, stanozolol, deoxymethyltestosterone (Madol), tetrahydrogestrinone, methandrostenolone (Metandienon), but also estrogens such as ethinyl estradiol, mestranol, stilbestrol, moreover anti-hormones such as anti-androgens like cyproterone acetate, flutamide or bicalutamide, anti-estrogens such as faslodex, as well as selective estrogen receptor modulators such as raloxifen, tamoxifen, toremifen, bazedoxifen, lasofoxifen, and selective androgen receptor modulators like andarine.

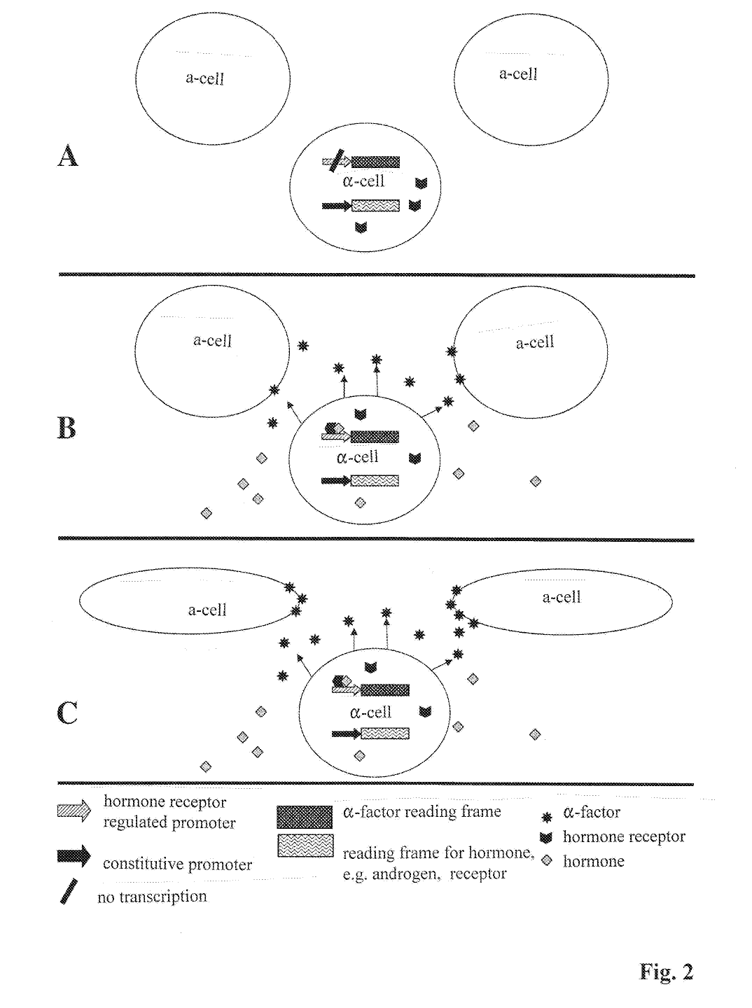

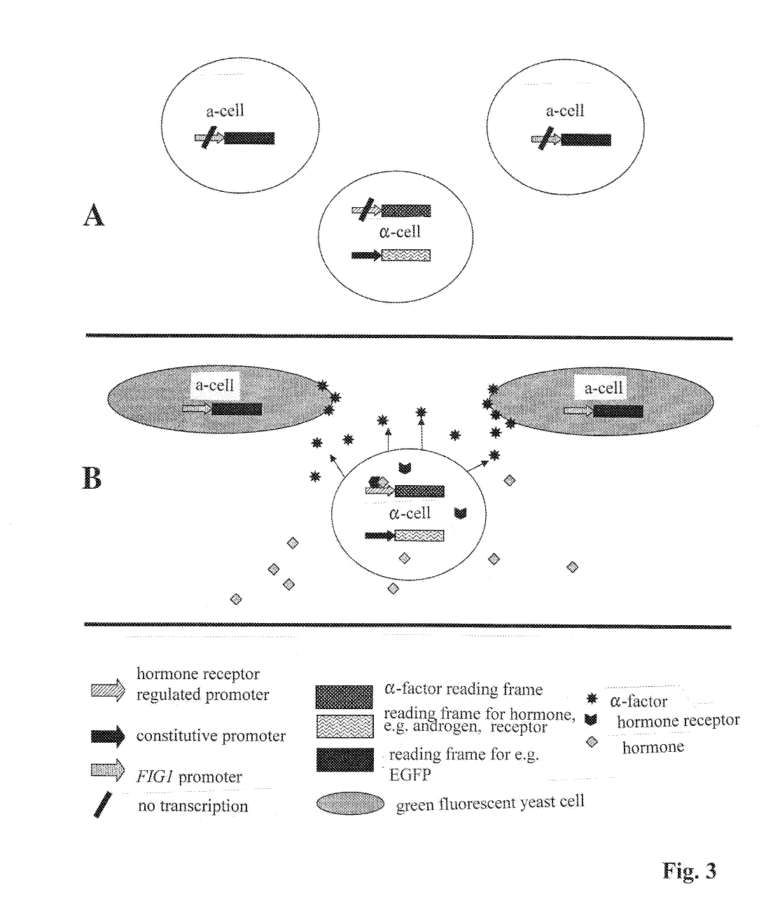

In the haploid yeast cells according to the invention the gene for the corresponding hormone receptor, for example, the androgen receptor or the estrogen receptor, is advantageously subjected to the control of a constitutive promoter in order to ensure the expression of the hormone receptor in the cell (FIG. 2; FIG. 3).

A promoter in the meaning of the invention is a DNA sequence that regulates the expression of a gene. In general, these regulating DNA regions are on the 5′ side of the transcription starting point of the corresponding gene. Such regulating regions can be close to the transcription starting point but can also be more than 1,000 bp remote from the coding sequence. They can also be positioned at the 3′ side of the coding sequence of the corresponding gene or within the transcribing sequence of the corresponding gene.

Promoters that are regulated by heterologous hormone receptors in the meaning of the present intention are preferably those regions of the genomic DNA that are specifically responsible for the regulation of the expression of a gene in that they react to the presence of hormonally active substances and, depending on these signals, activate or repress the expression of the gene under their control. When such promoters are positioned at the 5′ side of the transcription starting point of any gene, preferably a yeast pheromone gene, they regulate the activity of this gene as a function of the aforementioned hormone receptors.

Constitutive promoters in the meaning of the invention means that the reading frames or genes that are under the control of such a promoter are permanently expressed under the conditions of the method according to the invention. Preferred are those promoters that, independent of cell type, cell state and growth conditions, are expressed permanently.

The expression of heterologous hormone receptors in yeast cells is known in the prior art. For example, sensor systems for human hormones such as androgens (Bovee et al., 2008) can be expressed in yeasts. Preferred promoters for the constitutive expression of a target gene in yeast cells are, for example, the promoters ADH1, GPD, TEF2 and CYC1 (Mumberg et al., 1995). Further preferred promoters are listed in Table 2. Further constitutive promoters can be found in relevant databases, for example, the Saccharomyces Genome Database (SGD, Department of Genetics at the School of Medicine, Stanford University, U.S.A.) on the basis of the detected expression profiles.

TABLE 2
Select constitutive promoters in Saccharomyces cerevisiae

| Promoter | Sources |
|---|---|
| PGK1 | Protchenko et al., 2008 |
| | Sekler et al., 1995 |
| PGK1, SPT15 | McNabb et al., 2005 |
| PMA1 | Perez-Castineira et al., 2002 |
| | Opekarova et al., 1998 |
| SUF14 | Fairman et al., 1999 |
| | Hill et al., 1986 |
| GAPFL | Hottiger et al., 1994 |
| | Janes et al., 1990 |
| TPI1 | Chow et al., 1994 |
| TDH3 | Chernaik and Huang, 1991 |
| | Ecker et al., 1986 |

Promoters in the meaning of the invention are also DNA sections that are homologous to the corresponding yeast promoters that have a sequence identity of preferably more than 50%, preferably more than 80%. These sections can, for example, originate from homologous genomic regions of other organisms, preferably other yeast strains. However, they can also be synthetically produced DNA sequences whose sequence exhibits an identity of preferably more than 50%, preferably more than 80%, with the corresponding Saccharomyces cerevisiae promoter.

Promoters can also be synthetic DNA sequences that are combined of a partial region of one of the above-mentioned yeast promoters or regulative DNA sections of other organisms as well as a known basal promoter of Saccharomyces cerevisiae. The basal promoter provides the DNA sequences required for binding to the transcription machinery while the regulative DNA sections react specifically to regulating signals. Such a basal promoter is preferably the basal promoter of the cytochrome c gene of Saccharomyces cerevisiae that is contained in a 300 bp fragment at the 5′ side of the start codon of the cytochrome c gene (Chen et al., 1994).

Promoters of synthetic DNA sequences can also contain several sections of an identical DNA sequence. This multiplication of a regulatory DNA section enables advantageously an increase of the sensitivity of the promoter relative to the regulating factors such as transcription factors or hormone receptors.

According to the invention the haploid cells are moreover genetically modified such that a gene that codes for a mating type specific yeast pheromone is subjected to the control of a promoter that is regulated by the heterologous hormone receptor (FIG. 2; FIG. 3). The term promoter is to be understood as defined above.

Promoters that are regulated by hormone receptors are characterized by binding sites for the hormone receptors in question, so-called hormone response elements (HREs) such as androgen response elements (ARE) or estrogen response elements (ERE). Especially preferred is the derivative of the PGK promoter with 3 AREs (SEQ ID No. 1) described by Sohoni & Sumpter (1998).

SEQ ID No. 1
AACGAGTGTTTCCCTCCTTCTTGAATTGATGTTACCCTCATAAAGCAC
GTGGCCTCTTATCGAGAAAGAAATTACCGTCGCTCGTGATTTGTTTGC
AAAAAGAACAAAACTGAAAAAACCCCGGATCGGTACAAAATGTTCTAG
GTACAAAATGTTCTCGGTACAAAATGTTCTGAGCTCAAAGCGGCCGCG
ATCCGGTCGTCACACAACAAGGTCCTAGCGACGGCTCACAGGTTTTGT
AACAAGCAATCGAAGGTTCTGGAATGGCGGGAAAGGGTTTAGTACCAC
ATGCTATGATGCCCACTGTGATCTCCAGAGCAAAGTTCGTTCGATCGT
ACTGTTACTCTCTCTCTTTCAAACAGAATTGTCCGAATCGTGTGACAA
CAACAGCCTGTTCTCACACACTCTTTTCTTCTAACCAAGGGGGTGGTT
TAGTTTAGTAGAACCTCGTGAAACTTACATTTACATATATATAAACTT
GCATAAATTGGTCAATGCAAGAAATACATATTTGGTCTTTTCTAATTC
GTAGTTTTTCAAGTTCTTAGATGCTTTCTTTTTCTCTTTTTTACAGAT
CATCAAGGAAGTAATTATCTACTTTTTACAACAAATACAAAAGATCTG
CTAGCAAAA

Illustrated here is the sequence of the derivative of the PGK promoter: Sequence of the PGK promoter (italics) as well as of the three androgen response elements (AREs) (bold print) in the p426PGK vector.

Preferably, the PGK promoter is also used in order to generate by combination with other hormone response elements new promoters that are regulated by other hormone receptors than the androgen receptor. For this purpose, for example, the AREs contained in the PGK promoter derivative (SEQ ID No. 1) can be exchanged for other appropriate responsive elements.

The activation of the promoter that is regulated by the heterologous hormone receptor leads to the expression and secretion of the yeast pheromone. The yeast pheromones play an important role in yeast mating. Yeast cells basically can be present in the diploid state as well as in the haploid state. Two haploid yeast cells can fuse in a process that is referred to as yeast mating to a single diploid yeast cell. In case of haploid yeast cells one differentiates between two so-called mating types. Only yeast cells with different mating types can be mated with each other. For example, in case of baker's yeast Saccharomyces cerevisiae these are the mating types a and a, in case of fission yeast Schizosaccharomyces pombe the mating types plus and minus.

The haploid yeast cells according to the invention are preferably Saccharomyces cerevisiae or Schizosaccharomyces pombe cells. Preferably, the haploid yeast cells are Saccharomyces cerevisiae cells of the mating type a or Saccharomyces cerevisiae cells of the mating type a.

The respective yeast cells form short peptides, so-called yeast pheromones in order to notify their environment of their own mating type. For example, Saccharomyces cerevisiae yeast cells of the mating type a secrete the pheromone α-factor and Saccharomyces cerevisiae yeast cells of the mating type a the pheromone a-factor. The cells, for example, the yeast cells, have on their surfaces receptors for the pheromones of the respective opposite mating type. For example, Saccharomyces cerevisiae cells of the mating type a are able to detect in their environment Saccharomyces cerevisiae cells of the mating type α, and vice versa.

Yeast pheromones in the meaning of the invention are in addition to the natural yeast pheromones occurring in the yeast cells also homologous or modified peptides or peptide analogues or other organic compounds that are capable of binding to the pheromone receptors of the yeast cells and to activate them. The DNA sequence that codes for the yeast pheromone can be either a natural gene that is contained in the genome of an organism or a synthetic gene sequence whose expression leads to production of a yeast pheromone or of a peptide that is homologous to a yeast pheromone and is capable of activating the pheromone receptors of yeast cells.

The genes that are responsible in the haploid yeast cells for the synthesis of the yeast pheromone are preferably the MFα1 or MFα2 gene of Saccharomyces cerevisiae that codes for the pheromone α-factor or the MFA1 gene or MFA2 gene of Saccharomyces cerevisiae that codes for the pheromone α-factor.

When, by means of the recombinant expressed heterologous hormone receptors, the haploid yeast cells detect hormonally active substances contained in the sample, the transcription of the promoter that can be regulated by the hormone receptor is induced so that the haploid yeast cells, as a response to the hormonally active substance, express the yeast pheromone and secret it into the environment (FIG. 2 B, FIG. 3 B). Upon activation of the androgen receptors by an androgenically active hormone, for example, the yeast pheromone that is under the control of a PGK promoter derivative that has three androgen-responsive elements would be expressed and secreted.

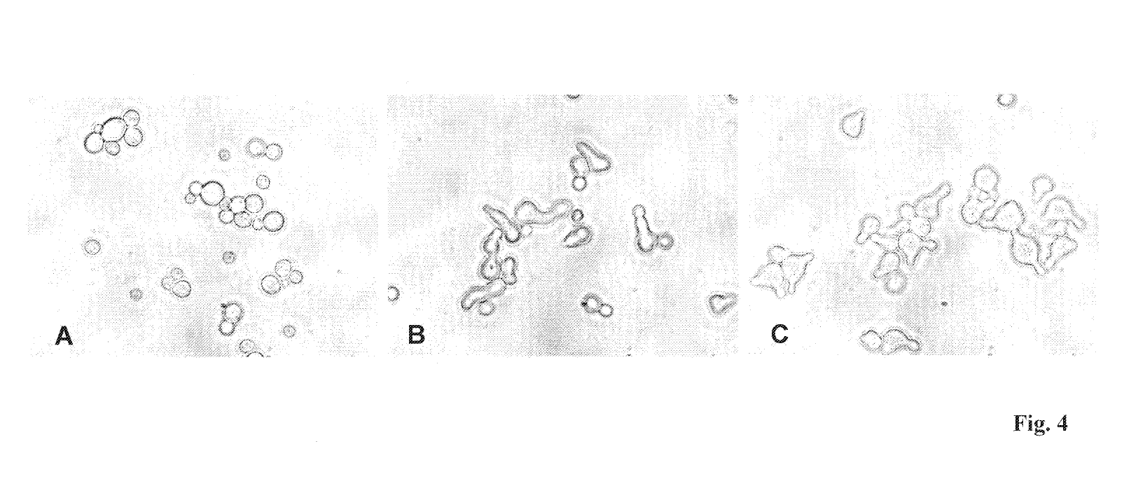

The haploid yeast cells according to the invention have on their surface receptors for the secreted yeast pheromone (feature 3). The activation of the receptors by the appropriate yeast pheromone triggers a complex genetic program which causes the change of the transcription activity of different promoters (for example, the very strong transcription increase (96 times) of the FIG1 promoter (Roberts et al., 2000) and a significant change of the morphology of the yeast cells (for example, “shmoo” effect) (FIGS. 4 B and C). The changed morphology can be easily detected by light microscope.

Therefore, preferably that mating type-specific pheromone is used for which the haploid yeast cell itself has receptors. The haploid yeast cells according to the invention are then uniform and comprise all features 1 to 3. For example, in a haploid Saccharomyces cerevisiae yeast cell of the a-type that has α-receptors on its surface, the gene for the α-factor is subjected to the control of a promoter that is regulated by the heterologous hormone receptor.

When this yeast cell expresses the heterologous hormone receptor, by binding of the hormonally active substance to the hormone receptor the expression and secretion of the α-factor is induced. The α-factor binds to the corresponding receptors of the haploid yeast cells and causes the above described morphological changes, for example, mating projections of the cells.

Alternatively, in a haploid Saccharomyces cerevisiae yeast cell of the α-type that expresses a heterologous hormone receptor, the gene for the a-factor is subjected to the control of a promoter that is regulated by the hormone receptor. This yeast cell has receptors for the α-factor and will generate also the morphological changes after binding of the hormonally active substance and the expression and secretion of the α-factor.

In another embodiment of the invention, in addition to the haploid yeast cells that are characterized by a heterologous hormone receptor and a gene that codes for a yeast pheromone and is under the control of a promoter that can be regulated by the hormone receptor, there are also haploid yeast cells of a second type that comprise the feature 3, i.e., a receptor for the yeast pheromone that is expressed and secreted by the haploid yeast cells according to the invention with the features 1 and 2. In this embodiment, the expression and secretion of this yeast pheromone advantageously causes an amplification of the signal that is triggered by a hormonally active substance. Moreover, as morphological changes not only the directed cell wall growth of the haploid yeast cells but also the fusion of haploid yeast cells to diploid yeast cells (zygote formation) can be detected.

The genes that naturally code for the yeast pheromones are preferably switched off in the utilized cells. It is preferred that the natural genes MFα1 and MFα2 in α-cells of Saccharomyces cerevisiae cells and the natural genes MFa1 and MFa2 in a-cells of Saccharomyces cerevisiae cells are deleted. In this way, it is advantageously ensured that the yeast pheromone is exclusively formed and secreted when the hormonally active substance to be detected is present. The means of switching off the gene activity in cells are known to a person of skill in the art. This is realized, for example, by “gene replacement” (Kaiser et al., 1994). This also allows advantageously for culturing haploid yeast cells of different mating types in a single culture without them fusing by yeast mating into diploid yeast cells.

The promoter that is regulated by the yeast pheromone is preferably the FIG1 promoter of Saccharomyces cerevisiae. On the basis of the detected expression profiles, a person of skill in the art can find further pheromone-induced promoters in relevant databases, for example, the Saccharomyces Genome Database (SGD), or can take them from publications.

The transcription of the FIG1 gene is increased up to 97 times after incubation of haploid yeast cells with pheromones of the respective opposite mating type (Roberts et al. (2000). It was originally found in a yeast “two hybrid screen” for identification of pheromone-regulated genes (Erdmann et al., 1998). The name FIG1 means “factor induced gene 1”. It is an integral membrane protein which is directly or indirectly participating in the Ca²⁺ uptake in the cell (Muller et al., 2003). After addition of the α-factor to a-cells, a significant increase of the protein after 60 minutes can be detected (Roberts et al., 2000). At the level of transcription an increase of the mRNA concentration by more than 97 times is observed already 20 minutes after addition of the respective opposite pheromone to the yeast cells (Roberts et al., 2000). The promoter of the FIG1 gene can therefore advantageously be highly regulated by the yeast pheromones of the respective opposite mating type. Accordingly, the action on yeast pheromones is quickly and sensitively detectable.

Preferably, as a promoter-containing region a DNA section is used that comprises up to 1,000 bp at the 5′ side of the FIG1 gene or a partial section of this DNA section that is capable of activating or suppressing, based on the presence of a yeast pheromone, the specific gene that is under the control of this sequence.

Especially preferred is the use of the DNA section that is obtained by PCR amplification of the Saccharomyces genome by using the primer Fig1-for (SEQ ID No. 2) and Fig1-rev (SEQ ID No. 3) (see Table 3).

TABLE 3
Primers for amplification of the FIG1 promoter.

| SEQ | | |
|---|---|---|
| ID | | |
| No. | Primer | Sequence (5′ → 3′) |
| 2 | Fig1-for | TAT TAT GAG CTC TTG AAT GAT CAA CCA |
| | | AAC GCC GAT AT |
| 3 | Fig1-rev | TAT TAT ACT AGT TTT TTT TTT TTT TTT |
| | | TTT GTT TGT TTG TTT GTT TGT TTA CTA |
| | | TAA |

The letters in bold print of the primers listed
in Table 3 delimit the utilized promoter region
of the FIG1 gene.
In italics the recognition sequences of the
restriction endonucleases SacI and SpeI are
identified which are used for cloning.

In the process of yeast mating the transcription factor Ste12p induces the expression of pheromone-responsive genes by binding of Ste12p to so-called “pheromone response elements” (PREs) in the promoter region of inducible genes (Dolan et al., 1989). Hagen et al. (1991) showed that tandem-like arranged PREs are sufficient in order to activate the pheromone-responsive expression of haploid specific genes in both mating types. PREs are elements of a length of 7 bp with the consensus sequence TGAAACA (Kronstad et al., 1987).

In the FIG1 promoter three putative binding sites for Ste12p have been identified (Harbison et al.; 2004).

The response time of the FIG1 promoter can be shortened by a higher number of PREs. For example, in addition to or in place of the authentic activator region of the gene a fragment of a length of 139 bp with the PREs of the regulatory region of FUS1 or a simple synthetic cluster of PREs can be used (Hagen et al., 1991). In this way, advantageously with a reporter construct of the modified FIG1 promoter and an EGFP marker a higher expression of the marker gene and improved responsiveness to reduced pheromone concentrations is achieved. Also, a temporally faster response of the system is achieved.

Preferably, in the haploid yeast cells that have a receptor for the yeast pheromone the transcription activator Ste12p is therefore overexpressed. The overexpression of STE12P causes an improved expression of pheromone-responsive genes, promoted by PREs (Dolan and Fields, 1990). Advantageously, by overexpression of the transcriptional activator Ste12p also the expression level of the specific gene under the control of the pheromone-dependent promoter is increased.

In order to bind to individual PREs, Ste12p requires partially further transcription activators such as the factor Mcm1p (Hwang-Shum et al., 1991). In the cells with the feature 3, especially Mcm1p is therefore overexpressed preferredly. The heterologous expression of this factor contributes also to increase of the expression of the specific gene that is under the control of the pheromone-dependent promoter.

For monitoring that is as prompt as possible, it may be advantageous to destabilize the marker proteins by addition of sequences that lead to an increased “turnover” of the proteins. In this way, the fluorescent protein that is used as a marker protein has a limited half-life. In this way, a quick response time upon decrease of transcription is ensured.

Such limited half-life can be achieved, for example, by modification of the N-terminal amino acid or the introduction of a signal sequence into the amino acid sequence of the marker protein coded by the specific gene so that the stability of the protein is lowered and its half-life is shortened. Preferably, for destabilization of the protein that is coded by the marker gene a so-called PEST domain is used that leads to a quick decomposition of the proteins by the ubiquitin system of the cell. Such PEST domains are known from many proteins. Preferably, the PEST domain of the G1 cyclin Cln2p of Saccharomyces cerevisiae is used. For this purpose, to the 3′ terminus of the coding sequence of the specific gene the coding sequence (SEQ ID No. 4) of the 178 carboxy-terminal amino acids of Cln2p (SEQ ID No. 5) and a stop codon are added.

Cln2p-Pest-Sequence (SEQ ID No. 4/5):
GCATCCAACTTGAACATTTCGAGAAAGCTTACCATATCAACCCCATCATGCTCTTTCGA
-A--S--N--L--N--I--S--R--K--L--T--I--S--T--P--S--C--S--F--E
AAATTCAAATAGCACATCCATTCCTTCGCCCGCTTCCTCATCTCAAAGCCACACTCCAA
--N--S--N--S--T--S--I--P--S--P--A--S--S--S--Q--S--H--T--P--
TGAGAAACATGAGCTCACTCTCTGATAACAGCGTTTTCAGCCGGAATATGGAACAATCA
M--R--N--M--S--S--L--S--D--N--S -V--F--S--R--N--M--E--Q--S-
TCACCAATCACTCCAAGTATGTACCAATTTGGTCAGCAGCAGTCAAACAGTATATGTGG
-S--P--I--T--P--S--M--Y--Q--F--G--Q--Q--Q--S--N -S--I--C--G
TAGCACCGTTAGTGTGAATAGTCTGGTGAATACAAATAACAAACAAAGGATCTACGAAC
--S--T--V--S--V--N--S--L--V--N--T--N--N--K--Q--R--I--Y--E--
AAATCACGGGTCCTAACAGCAATAACGCAACCAATGATTATATTGATTTGCTAAACCTA
Q--I--T--G--P--N--S--N--N--A--T--N--D--Y--I--D--L--L--N--L-
AATGAGTCTAACAAGGAAAACCAAAATCCCGCAACGGCGCATTACCTCAATGGGGGCCC
-N--E--S--N--K--E--N--Q--N--P--A--T--A--H--Y--L--N--G--G--P
ACCCAAGACAAGCTTCATTAACCATGGAATGTTCCCCTCGCCAACTGGGACCATAAATA
--P--K--T--S--F--I--N--H--G--M--F--P--S--P--T--G--T--I--N--
GCGGTAAATCTAGCAGTGCCTCATCTTTAATTTCTTTTGGTATGGGCAATACCCAAGTA
S--G--K--S--S--S--A--S--S--L--I--S--F--G--M--G--N--T--Q--V-
ATATAG
-I- *

The method according to the invention has the advantage that the signal caused by binding of a hormonally active substance to the heterologous hormone receptor can be amplified by a multiple as a result of the triggered secretion of the yeast pheromone and its effect on the surrounding cells so that advantageously the sensitivity is increased.

The α-factor is split by the specific protease Bar1p of Saccharomyces cerevisiae and is thus deactivated. Bart p is secreted and is required for correct mating of the yeast cells. MATa cells in which Bart p is deactivated show a significantly increased sensitivity relative to the α-factor. In order to increase the sensitivity and response time of the detection system, therefore cells are advantageously used in which the Bar1p protein is deactivated or the corresponding gene has been deleted (Ballensiefen and Schmitt, 1997; Chan and Otte, 1982; Barkai et al., 1998; Sprague and Herskowitz, 1981).

Moreover, in the haploid yeast cells preferably the protein Fig1p is deactivated. High local concentrations of the yeast pheromones cause cell death in yeast cells. By deactivation of Fig1p this effect is prevented (Zhang et al., 2006). In this way, it is advantageously prevented that the yeast cells by too high a concentration of yeast pheromone will die off and therefore would no longer be available for the method (Zhang et al., 2006).

Since the cells are in an aqueous medium for performing the method according to the invention, the hormonally active substances to be detected must also be present in this liquid medium.

The haploid yeast cells express according to the invention at least two heterologous genes: First the gene for the hormone receptor under the control of a constitutive promoter is expressed. The yeast pheromone gene that has been subjected to the control of a promoter that is specific for a hormone receptor is expressed by the activation of the hormone receptor.

Moreover, where appropriate, a specific gene that is under the control of the pheromone receptor and codes for a marker protein is expressed by the secretion of the yeast pheromone.

These genes must first be introduced into the haploid yeast cells. In this connection, they can be present in the haploid yeast cell on an extra-chromosomal DNA molecule. For this purpose, a yeast expression vector is preferably employed that upon division of the yeast cell is replicated and distributed stably onto the daughter cells. Especially preferred is a so-called “high copy number” vector that is present in the yeast cell in a large number of copies. Alternatively, also vectors are used that are present in smaller copy numbers or as individual vectors in the yeasts, for example, ARS-CEN vectors or artificial yeast chromosomes (yeast artificial chromosomes).

In another embodiment, the gene in question is integrated together with the regulated promoter into the chromosomal DNA of the haploid yeast cell. In this way, it is advantageously ensured that all offspring of the haploid yeast cell also contain the marker genes under the control of the specific promoter.

Advantageous for using the method is also that yeasts can be dried well and therefore can be kept for an extended period of time. For example, an inexpensive air drying method has been developed in which first wheat flour is added to a moist yeast culture and then air drying is performed. With this method, survival rates of the cells of up to 100% have been achieved (Mille et al., 2005). Air-dried yeasts can advantageously be transported and stored without any great expenditure.

In a special embodiment of the method according to the invention the haploid yeast cells can also be immobilized. Hydrogels of calcium alginate are suitable, for example, as a matrix for embedding. They have a porous structure and are optically transparent. These properties enable, on the one hand, the interaction between the immobilized cells and their surroundings, on the other hand, the detection of optical signals that are generated by the haploid yeast cells, for example, fluorescent proteins. Methods for embedding of Saccharomyces cerevisiae yeast cells in the form of alginate spheres for the development of applications in the field of biosensing/biotechnology are known in the literature (Belve et al., 2008; Fine et al.; 2006). In analogy, immobilization of the cells in thin transparent calcium alginate layers is conceivable.

The method according to the invention and the haploid yeast cells according to the invention can be used in numerous ways. An aspect of the invention is therefore also the use of the method according to the invention or of the inventive yeast cells for diagnostics, i.e., for detecting hormonally active substances in urine samples, blood samples, and serum samples, especially for detecting doping agents in urine samples. The method according to the invention or the yeast cells according to the invention are suitable also for use in estimating loading of environmental samples with hormonally active substances, such as water samples and tissue samples, or for evaluating the risk potential of possibly hormonally active synthetic or natural substances. The method according to the invention or the yeast cells according to the invention are suitable in this connection in particular for the inexpensive and fast screening of large numbers of substances or complex substance mixtures. In this connection, they are suitable in particular for identification of potentially hormonally active substances for pharmaceutical application, for example, from plant extracts or in case of unknown substances of plant or other organic or synthetic origin. Moreover, the method according to the invention is also suitable for use in toxicological screening of wastewater, drinking water, packaging materials, foodstuffs or food supplements with respect to undesirable hormonal ingredients. Also preferred is the use for applications in screening for use of hormones in fattening animals that are to be used for producing foodstuffs.

With the aid of the following figures and embodiments the invention will be explained in more detail without being limited thereto.

It is shown in:

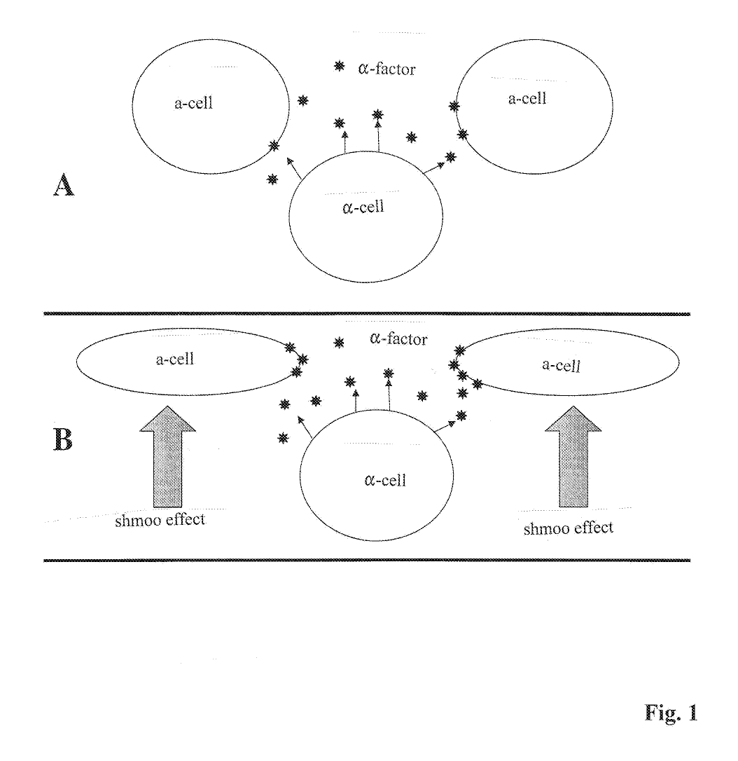

FIG. 1: schematic illustration of a yeast pheromone system;

FIG. 2: schematic illustration of the method for detecting hormonally active substances by detection of morphological changes of the haploid yeast cells;

FIG. 3: schematic illustration of the method for detecting hormonally active substances by detecting physiological changes of the haploid yeast cells;

FIG. 4: light microscopic illustration of the “shmoo” effect. To the left Saccharomyces cerevisiae cells of the mating type a are shown that divide by budding by mitosis (FIG. 4 A). The images at the center (FIG. 4 B) and to the right (FIG. 4 C) show the morphological changes (“shmoo” effect) caused by the action of a yeast pheromone (α-factor); magnification 1,000 times;

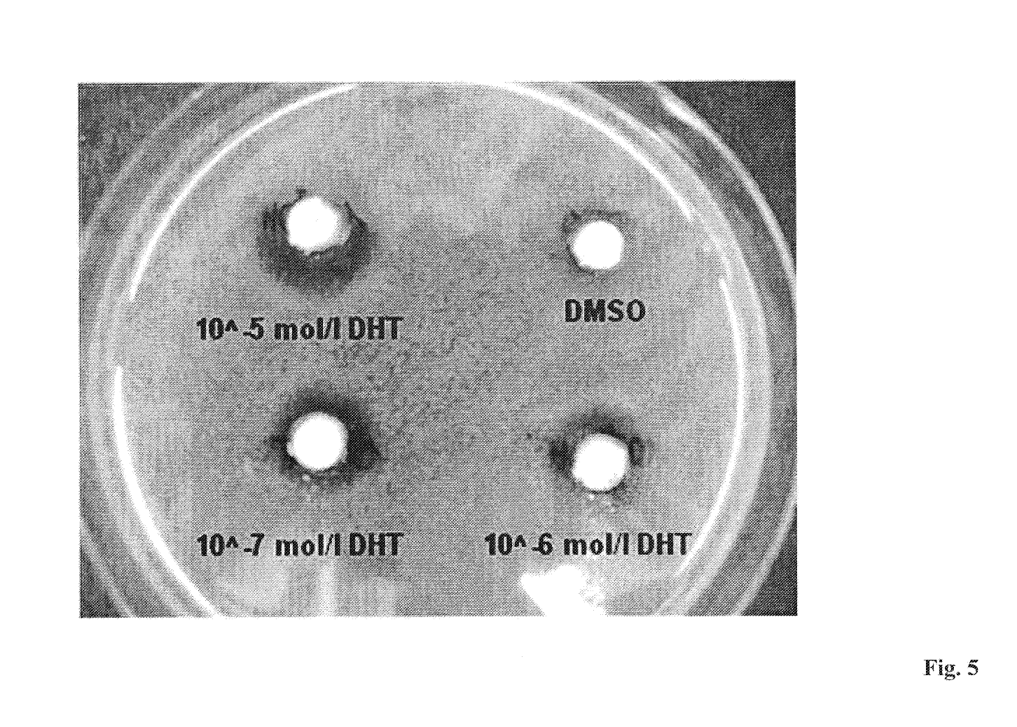

FIG. 5: so-called HALO assay by use of yeast strains BY4741 Δbar1 (in agar) and BY4741 Δbar1 p423GPD-AR1, p426PGK-MFα1 (on filter plates), use of dimethyl sulfoxide (DMSO) as negative control and different DHT concentrations;

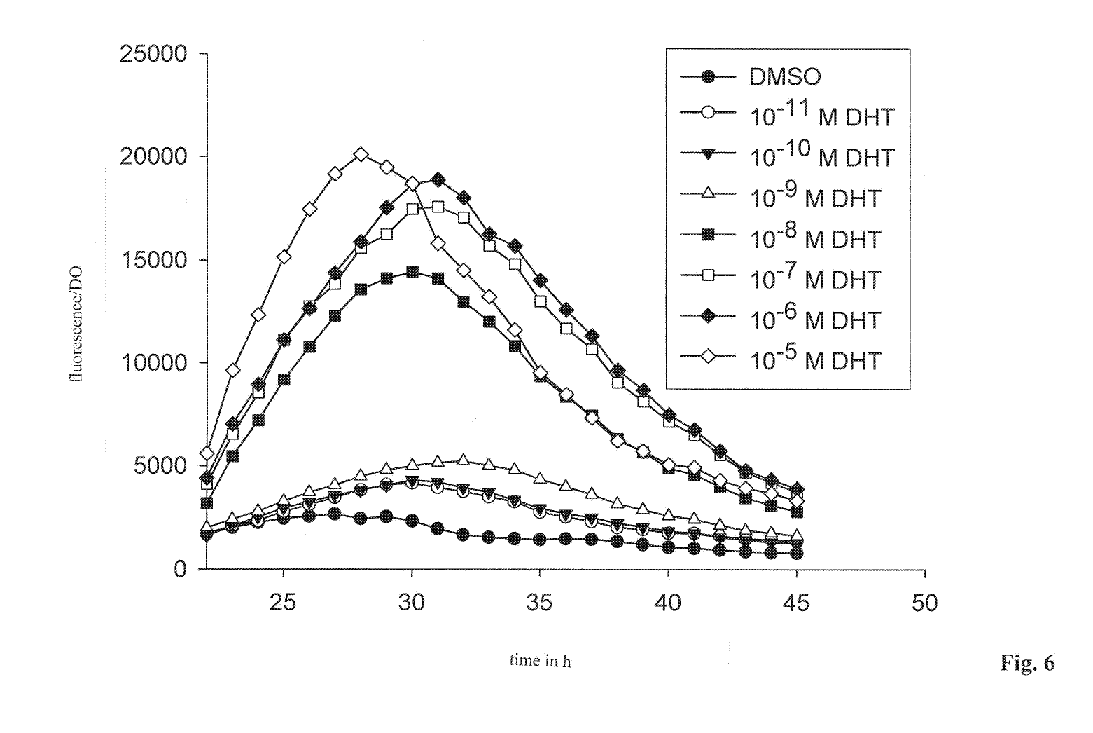

FIG. 6: illustration of the quotient of fluorescence signal (EGFP) and optical density for 10⁻¹¹-10⁻⁵ mole dihydro testosterone (DHT) in the incubation period 22-45 hours at 30° C. for the yeast strain BY4741 Δbar1 p423GDP-AR1, p426PGK-EGFP.

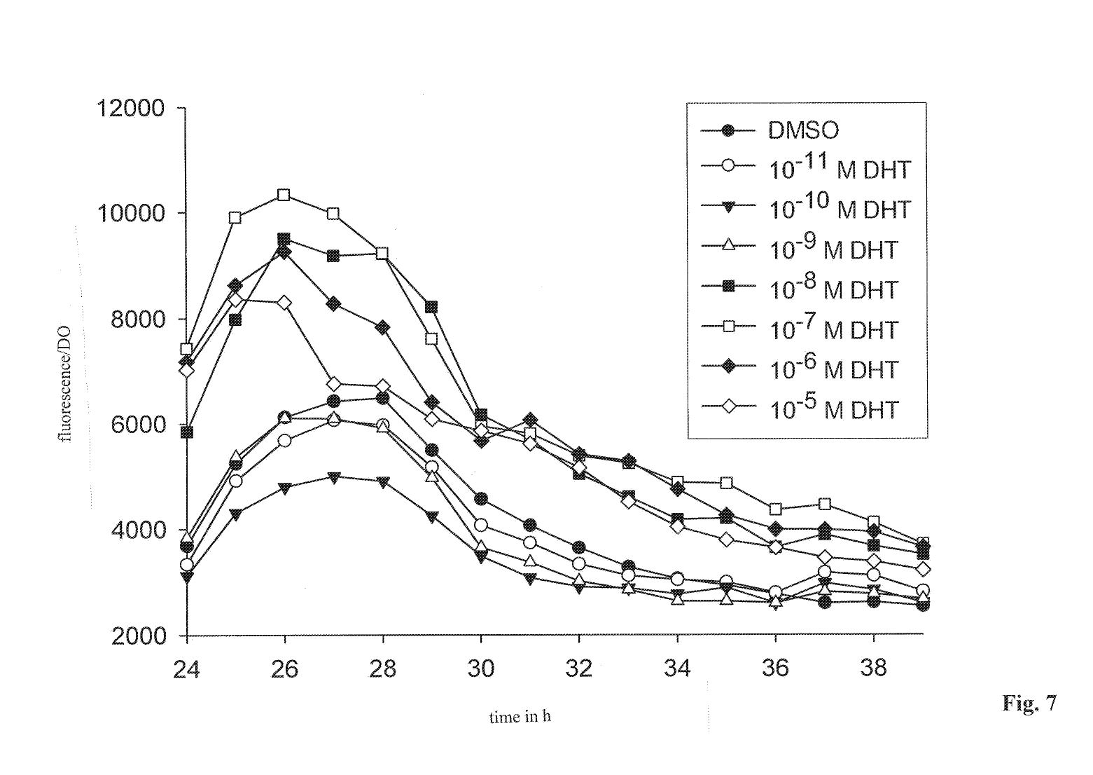

FIG. 7: illustration of the quotient of fluorescence signal (EGFP) and optical density for 10⁻¹¹-10⁻⁵ mole DHT in incubation period 24-39 hours at 30° C. yeast strain BY4741 p423GDP-AR1, p426PGK-EGFP.

### EXAMPLE 1

### Generation of Yeast Cells that Express the Human Androgen Receptor (AR)

In order to express the androgen receptor constitutively in the yeast, the plasmid p423GPDAR1 was constructed. For this purpose, the AR1 reading frame (GeneID: 367 Accession No. NP_{—}000035) was cloned into the plasmid p423GPD as follows (Mumberg et al., 1995).

By means of the primers hAR1 BcuI(SpeI)_f2 (SEQ ID No. 6; TATATAACTAGTATGGAAGTGCAGTTAGGGCT) and hAR1SalI_rev (SEQ ID No. 7; ATATATGTCGACTCACTGGGTGTGGAAATAGATG) the AR1 gene sequence (2,763 bp) was PCR-amplified. By means of the primers used in the PCR, to the AR1 reading frame at the 5′ end the SpeI cutting site and at the 3′ end the sequence of the SalI cutting site were added. The PCR batch was purified for further use.

The p423GPD vector and the purified PCR product for the AR1 reading frame were cut by the restriction enzymes SpeI and SalI. Subsequently, the purified cut vector p423GPD and the purified cut PCR product for the androgen receptor were separated by means of gel electrophoresis on a 1% agarose gel. The specific fragments were cut out of the gel and the DNA contained therein was extracted from the piece of agarose gel. This was followed by ligation of the AR1 fragment and the p423GPD plasmid. After completed electroporation, the batches were placed onto LB medium plates with 100 μg/ml ampicilin. Recombinant clones were identified by a control digestion of isolated plasmid DNA with the restriction enzyme KpnI.

In order to be able to check the correct position of the AR1 reading frame in the MCS, the plasmid DNA was sequenced. The sequencing with the GPDpromseqfor primer (SEQ ID No. 8; CGGTAGGTATTGATTGTAATTCTG) showed that the AR1 gene was integrated at the correct site in the MCS.

### EXAMPLE 2

### Generation of Yeast Cells that Contain a Gene for the α-Factor that is Under the Control of an Androgen-Inducible Promoter

The MFα1 reading frame that codes for the α-factor was cloned behind an androgen sensitive promoter into the plasmid p426PGK (see below).

The promoter was used in the androgen-inducible yeast expression strain PGKhAR.

The yeast strain PGKhAR has been described by Purvis et al. (1991). In this strain the DNA sequence for the human androgen receptors has been integrated stably into the genome. The androgen receptor is expressed constitutively. In addition, the yeast strain comprises a plasmid that carries a derivative of the PGK promoter with three androgen-responsive elements and the lacZ gene as a reporter gene. Since the PGK promoter is a derivative, it is not constitutively expressed in the yeast cells. In the presence of androgens, the androgen receptor that is activated by the ligand binding will bind on the PGK promoter derivative and initiate in this way the transcription of the reporter gene IacZ. The expression of the reporter gene is realized at a high level. After translation the β-galactosidase is secreted by the yeast cell. The activity of the enzyme can be measured photometrically at 540 nm because the enzymatic substrate conversion produces a colored product (Sohoni & Sumpter, 1998; Routledge & Sumpter, 1996).

Cloning of the MFα1 reading frame was realized as follows. For multiplication of the MFα1 reading frame (GeneID:855914 Acc. Number X01581), the primer pair MFalpha1SpeI_for/MFalpa1BamHI_re (SEQ ID No. 9; TATATTACTAGTATGAGATTTCCTTCAATTTTTACTG1 SEQ ID No. 10 TATATTGGATCCTTAGTACATTGGTTGGCCG) and the plasmid p426GPDMFα1 as a template were used. p426GPDMFα1 is based on the vector p426GPD (Mumberg et al., 1995) into which the MFα1 reading frame was inserted by means of the restriction cutting sites EcoRI and SalI.

By means of the primers selected for PCR, to the MFα1 gene at the 5′ end the sequence of a SpeI cutting site and at the 3′ end the nucleotide sequence of a BamH1 cutting site were added. The PCR product was analyzed by means of agarose gel electrophoreses and subsequently purified.

The MFα1 reading frame was cloned into the vector p426PGK that is based on the vector p426ADH (Mumberg et al., 1995). The vector p426ADH was first digested with the restriction enzyme SacI and subsequently the ADH1 promoter was removed. Since SacI also has a cutting site in the PGK promoter, the projecting 3′ ends of the vector were removed by means of S1 nuclease. This leads to blunt end generation. The PGK promoter derivative (SEQ ID No. 1) was amplified by PCR. As a template the plasmid described by Sohoni & Sumpter (1998) was used which is a derivative of the PGK promoter with 3 AREs. By digestion of the PCR product with the restriction endonuclease DraI a ligation with the blunt ends of the cut p426ADH plasmid was enabled. Subsequently, the PGK PCR product and the processed p426ADH plasmid was digested with SpeI. Accordingly, the ADH1 promoter was cut from the plasmid and compatible projecting ends generated on the PCR product. This is followed by ligation of the derivative of the PGK promoter with 2 AREs (Sohoni & Sumpter, 1998) (SEQ ID No. 1) into the p246 plasmid.

The purified PCR product for the MFα1 gene and the p426PGK vector were digested with the restriction enzyme BamH1 and SpeI. For the ligation a molar ratio vector:insert of approximately 1:9 was selected. The ligation was done by 4° C. over night.

The ligation batch for the plasmid p426PGKMFα1 was transformed into the E. coli strain DH10B. The transformation batch was placed onto LB plates with 100 μg/ml ampicillin and cultured over night at 37° C.

For identification of recombinant plasmids a PCR of the transformands with the primer pair MFalphalSpeI_for/ MFalpa1BamHI_re was carried out. The complete MFα1 reading frame was sequenced in a recombinant plasmid with the MFfor primer (SEQ ID No. 11; TCGTAGTTTTTCAAGTTCTTAGATGC). Sequencing showed that no mutations were present. The thus generated vector p426PGKMFα1 was used for the following experiments.

### EXAMPLE 3

### Inactivation of the Chromosomal Genes that Code for Yeast Pheromones

Advantageously, for the inventive method yeasts are used whose authentic expression of yeast pheromones is deactivated. For this purpose, the corresponding genes can be deleted. As an example, the deactivation of the genes MFα1 and MFα2 is disclosed that both code for the α-factor. Yeast cells that are modified in this way no longer are capable of forming the α-factor endogenously.

For the deletion of the reading frame MFα1 and MFα2, for example, in the α-yeast strain BY4742 (MATα, his3Δ1, leu2Δ0, lys2Δ0, ura3Δ0), the marker cassettes natMX6 and hphMX6 are used which impart resistances against the antibiotics nourseothricin or hygromycin B. The natMX6 cassette is amplified by SFH PCR by means of primer SEQ ID No. 12 and SEQ ID No. 13 of Table 4. The 5′ regions of the primer (50 bases each) are homologous to the flanking sequences of the MFα1 reading frame in the genome of Saccharomyces cerevisiae. The 3′ regions of the primer (20 bp) are homologous to the ends of the natMX6 cassette. As DNA templates for the SFH PCR the plasmid pFA6a-natMX6 (Hentges et al. 2005) is used. Subsequently, yeast cells are transformed with the SFH fragment. Transformands in which the fragment is integrated stably into the genome by double homologous recombination are selected on a medium that contains nourseothricin and the correct integration of the deletion cassette was confirmed by means of diagnostic PCR. Subsequently, the deletion of the reading frame MFα2 in the generated Δmfα1 yeast strain was carried out. For this purpose, in analogy to the first deletion an SFH fragment with the primer SEQ ID No. 14 and SEQ ID No. 15 (see Table 4) and the hphMX6 cassette (DNA templates pFA6a-hphMX6, Hentges et al., 2005) is amplified and transformed in Δmfα1 yeast cells. The 5′ side regions of the primer are homologous to the flanking sequences of the MFα2 reading frame in the genome of Saccharomyces cerevisiae. The selection of positive transformands is realized by hygromycin B-containing medium and the correct integration of the hygromycin B resistance cassette in the Δmfα1-Δmfα2 yeast strain is checked by means of diagnostic PCR.

TABLE 4
Primers for the deletion of the reading frame of
MFa1 and MFa2 of Saccharomyces cerevisiae.

| SEQ | | |
|---|---|---|
| ID | | |
| No. | Name | Sequence (5′ → 3′) |
| 12 | MFalp1_F2 | AAGAAGATTACAAACTATCAATTTCATACAC |
| | | AATATAAACGATTAAAAGACGGATCCCCGGG |
| | | TTAATTAA |
| 13 | MFalp1_R1 | TGGGAACAAAGTCGACTTTGTTACATCTACA |
| | | CTGTTGTTATCAGTCGGGCGAATTCGAGCTC |
| | | GTTTAAAC |
| 14 | MFalp2_F2 | TTACTACCATCACCTGCATCAAATTCCAGTA |
| | | AATTCACATATTGGAGAAACGGATCCCCGGG |
| | | TTAATTAA |
| 15 | MFalp2_R1 | ATGAACGTGAAAGAAATCGAGAGGGTTTAGA |
| | | AGTAGTTTAGGGTCATTTTGAATTCGAGCTC |
| | | GTTTAAAC |

The unmarked prima sequence characterizes areas
which are homologous to genomic DNA of
Saccharomyces cerevisiae. The regions that are
homologous to the deletion cassette are shown
in bold print.

The natural genes MFα1 and MFα2 that code for the pheromone a-factor are deactivated in analogy to the above procedure. The production of strains in which all genes that endogenously code for the yeast pheromones are deleted is realized in the genetic system yeast advantageously by means of tetrad analysis. For example, the double mutants can be identified unambiguously in the cross of two individually hygromycin-resistant strains in the tetrad of the so-called “non-parental ditype”. Accordingly, strains are produced whose genes MFa1, MFa2, MFα1 and MFα2 are deleted.

### EXAMPLE 4

### Generation of Yeast Cells for Detecting Androgenic Substances by Means of Cell-Morphological Changes (“Shmoo” Effect)

A yeast strain, e.g. W303 (Mat a, ade2-1, his31, his3-15, leu2-3, leu2-112, trp1-1, ura3-1), in which the BAR1 gene has been deleted is transformed with the plasmids p426PGKMFα1 and p423GPDAR1. The plasmid p426PGKMFα1 carries for selection in yeast the URA3 gene. Yeasts that because of genomic ura3 mutations require uracil do no longer require the addition of uracil after transformation with the plasmid. The plasmid p423GPDAR1 can be selected by means of the H1S3 gene. Appropriate histidine and uracil prototrophic transformands are selected. The expression of the androgen receptors in the cells is verified by means of Western blot analyses. The induction of morphological changes by the α-factor is microscopically documented.

The thus produced strain is mixed with the strains of the mating type a that are furnished with the vector p426FIG1-EGFP (see Example 6). The induction of expression and secretion of the α-factor in the transformed W303 cells causes in cells that are transformed with p426FIG1-EGFP enhanced formation of EGFP, which can be accordingly read out. Where appropriate, such mixed cultures of different transformed cell types can be used for signal amplification.

### EXAMPLE 5

### Deactivation of the BAR1 Gene in Yeast Cells for Increasing the Sensitivity of the Detection Method

For the method according to the invention advantageously yeasts are used in which the protease Bar1p is deactivated that decomposes receptor-bound α-factor on the cell surface of Saccharomyces cerevisiae. Preferably, for this purpose the BAR1 reading frame is deleted by means of the marker cassette bleMX6. The cassette imparts resistance against the antibiotic phleomycin.

For this purpose, by SFH-PCR with primers SEQ ID No. 16 and SEQ ID No. 17 in Table 5 the bleMX6 marker cassette is amplified by plasmid pFA6-bleMX6 (Hentges et al., 2005). The 50 bases in the 5′ region of the two primers are homologous to the flanking sequences of the BAR1 reading frame, the 20 bases of the 3′ region of the two primers are homologous to the flanks of the bleMX6 cassette. Subsequently, yeast cells, for example, Saccharomyces cerevisiae cells with the genotype MATa ΔMFa1, ΔMFa2, are transformed with the SFH fragments in order to delete the BAR1 reading frame by double homologous recombination with the marker cassette. The selection of positive transformands is realized on phleomycin-containing medium. The correct genomic integration is verified by means of diagnostic PCR.

The production of further yeast strains with deleted BAR1 is realized in analogy to Example 4 with the described tetrad analysis.

TABLE 5
Primers for the deletion of the reading frame
of the protease Barlp in Saccharomyces cerevisiae
with a marker cassette bleMX6.

| SEQ | | |
|---|---|---|
| ID No. | Name | Sequence (5′ → 3′) |
| 16 | deltaBar1_F2 | ATCGCCTAAAATCATACCAAAATAAAAA |
| | | GAGTGTCTAGAAGGGTCATATACGGATC |
| | | CCCGGGTTAATTAA |
| 17 | deltaBAR1_R1 | ACTATATATTTGATATTTATATGCTATA |
| | | AAGAAATTGTACTCCAGATTTCGAATTC |
| | | GAGCTCGTTTAAAC |

The unmarked primer sequence characterizes regions
that are homologous to genomic DNA of Saccharomyces
cerevisiae. Regions that are homologous to the
bleMX6 deletion cassette are shown in bold print.

### EXAMPLE 6

### Generation of Haploid Yeast Cells that Contain a Marker Protein Under the Control of a Promoter that can be Controlled by a Yeast Pheromone

The Saccharomyces cerevisiae yeast cells of the mating type a that are present in the same batch as cells of the second type are modified in that they contain the reading frame coding for EGFP under the control of the FIG1 promoter.

For this purpose, by using the primer Fig1-for (SEQ ID No. 18) and Fig1-rev (SEQ ID No. 19) (see Table 6), 1,000 bp at the 5′ side of the open reading frame of FIG1 were amplified by PCR, purified, cut with the restriction endo nucleases SacI and SpeI and cloned into the Saccharomyces cerevisiae vector p426. The thus produced vector (p426FIG1) was cut with the enzymes SalI and EcoRI.

The reading frame that codes for EGFP was PCR-amplified by means of primer EGFPEcofor and EGFPSalrev and the fragment of 744 bp was cut with the enzymes Sail and EcoRI, purified, and used for ligation into the vector p426FIG1.

TABLE 6
Primers for amplification of the EGFP gene.

| SEQ ID | | |
|---|---|---|
| No. | Primer | Sequence |
| 18 | EGFP | TAT TAT GAA TTC ATG GTG AGC AAG |
| | Ecofor | GGC GAG GAG |
| 19 | EGFP | TAT TAT GTC GAC TTA CTT GTA CAG |
| | Salrev | CTC GTC CAT GCC G |

The letters shown in bold print delimit the coding
reading frame of the EGFP gene.
In italics the recognition sequences of the
restriction endonucleases EcoRI and SalI are
indicated that are used for cloning into the
vector p426FIG1.

The DNA sequence of the cloned reading frame was verified by DNA sequence analysis. Thus, the vector p426FIG1-EGFP for the transformation of yeast cells was made available.

When after induction of the specific regulating promoter the then formed and secreted α-factor reaches surrounding Saccharomyces cerevisiae yeast cells of the mating type a, in these cells the transcription of the reading frame coding for GFP is strongly induced by means of the FIG1 promoter. This results in green fluorescence of the yeast cells which can be read out by sensor technology. The intensity of the green fluorescence can be proportional to the number of the a-cells surrounding the α-cells.

### EXAMPLE 7

### HALO Assay

By using the method according to the invention a modified HALO assay is used for determining the hormone/androgen concentration of a solution.

The so-called HALO assay is usually employed as a pheromone-based testing system on agar plates for determining the mating type of a yeast strain. Depending on the mating type, S. cerevisiae strains form and secrete the a-factor or α-factor as a pheromone. The recognition of the peptide of the respective other mating type, by means of surface receptors leads inter alia to the arrest of the cells in the G₁ phase of the cell cycle, i.e., these cells do not grow or grow very slowly.

For detection of the androgen concentration of a solution, first the yeast strains BY4741 Δbar1 in YPD medium and BY4741 Δbar1 p423GPD-AR1, p426PGK-MFα1 in W0 medium with leucine and methionine are incubated over night at 30° C. while being shaken. The yeast strain BY4741 Δbar1 should reach the plateau phase of the growth curve. For the yeast strain BY4741 Δbar1 p423GPD-AR1, p426PGK-MFα1 an optical density of OD₆₉₀ₙₘ=1 is sufficient.

YPD agar (0.5%) is heated and cooled to 55° C. To 25 ml each of liquid YPD agar 10 μl of the yeast strain BY4741 Δbar1 are added. Subsequently, the agar is polymerized in Petri dishes. While this is happening, the overnight culture of the yeast strain BY4741 Δbar1 p423GPD-AR1, p426PGK-MFα1 is centrifuged and the pellet is taken up in 1.5 ml medium. From this cell suspension 100 μl each are used and different DHP concentrations or another hormone is added, respectively. Dimethyl sulfoxide (DMSO) is used as a negative control. 20 μl of the hormone-containing cell suspension are placed onto sterile filter plates. The latter are placed onto solidified agar plates. The incubation of the agar plates is done at 30° C. in an incubator for 48 hours.

When the yeast strain BY4741 Δbar1 p423GPD-AR1, p426PGK-MFα1 is incubated with an androgen, the constitutively expressed androgen receptor recognizes the androgen and induces the expression of the MFα1 gene. The thus formed α-factor is secreted and is recognized by the yeast strain BY4741 Δbar1 by surface receptors because it is of the mating type a. Thus, in the presence of androgens growth inhibition occurs which can be recognized because of zones of inhibition on the agar plate.

The result documentation is realized by photographing the agar plates and measuring the resulting zones of inhibition. As an example, in this connection the following illustration (compare FIG. 5) is to be shown. A significant difference with regard to the size of the zone of inhibition between the negative control and the different androgen concentrations can be seen. Accordingly, by means of the HALO assay a qualitative and a semi-quantitative statement in regard to the androgen contents of a solution can be made.

### EXAMPLE 8

### Analysis of Androgenic Properties of Substances by Means of a Reporter Gene (EGFP) in Saccharomyces cerevisiae

The yeast strains to be used have identical auxotrophies in order to avoid a loss of plasmid for common incubation. In this case, the leucine and methionine auxotrophies of the yeast strains are used.

As a positive control first a yeast strain is tested that expresses constitutively the hormone receptor as well as the EGFP gene under the control of the androgen-sensitive promoter. For this purpose, the yeast strains BY4741 Δbar1 p426PGK-EGFP, p423GPD-AR1 and BY4741 Δbar1 p426PGK, p423GPD are cultured with addition of different concentrations of the androgen DHT (dihydro testosterone). DHT is taken up by the yeasts and binds intracellularly on the androgen receptor Isoform1(AR1) that is constitutively formed under the control of the GPD promoter. The receptor ligand complex binds in turn on the androgen-responsive elements in the PGK promoter of the p426PGK-EGFP plasmid and induces the expression of the EGFP gene. Thus, in the presence of androgenically active substances a fluorescence signal without amplification effect can be detected.

For testing the functionality of the yeast strain BY4741 Δbar1 p426PGK-EGFP, p423GPD-AR1 an incubation of the yeast cells in a 96 well plate was performed. For this purpose, 5×10⁴ cells/well in 100 μl W₀ medium with leucine and methionine in the presence of different androgen concentrations were incubated for 0 to 45 hours at 30° C. The measurement of EGFP signal was done hourly in the time frame of 22 hours-45 hours of incubation at an excitation wavelength of 485 nm and emission wavelength of 535 nm. As a reference the adsorption at 690 nm is determined. In an exemplary fashion, in this connection the EGFP signal in the presence of different DHT concentrations is illustrated (compare FIG. 6).

A clear EGFP signal was observed beginning with a DHT concentration of 10⁻⁸ mole. Lower androgen concentrations do not differ significantly from the negative control (DMSO=dimethyl sulfoxide). An incubation time of 30 hours has been found to be an optimal time for carrying out a measurement. The experiment shows that the yeast strain BY4741 Δbar1 p426PGK-EGFP, p423GPD-AR1 can be induced with DHT and thus can be used for testing the amplification system. Further measurements showed that the EGFP formation can also be triggered by further androgenically active substances.

As a negative control first the yeast strains BY4741 Δbar1 p423GDP-AR1, p426PGK and BY4741 Δbar1 p426FIG1-EGFP, p423GPD in the presence of different androgen concentrations were incubated together. The yeast strain BY4741 Δbar1 p423GDP-AR1, p426PGK recognizes androgenically active substances because the androgen receptor is constitutively formed. This yeast strain produces however no α-factor because the MFα1 gene is missing. Accordingly, the EGFP expression cannot be induced in the yeast strain BY4741 Δbar1 p426FIG1-EGFP, p423GPD. No EGFP signal should be measurable. Based on this control, an α-factor-independent induction of the EGFP expression can be excluded.

A further negative control is constituted by the common culturing of the yeast strains BY4741 Δbar1 p423GPD, p426PGK-MFα1 and BY4741 Δbar1 p426FIG1-EGFP, p423GPD. Based on this control, an induction of the MFα1 expression independent of binding of the receptor ligand complex can be checked. Because of the missing AR1 gene no androgen receptor where androgenically active substances can bind exists in the yeast cells. Therefore, the expression of the MFα1 gene cannot be induced. As a result of the missing α-factor the formation of EGFP is not possible.

For using the amplification system where two different haploid yeast cells are used, the yeast strains BY4741 Δbar1 p426PGK-MFα1, p423GPD-AR1 (sensor strain) and BY4741 Δbar1 p426FIG1-EGFP, p423GDP (reporter strain) are incubated together in the presence of different androgen concentrations. According to the amplifier system according to the invention, EGFP is formed in the presence of androgenically active substances.

Performing the experiment is possible with, in addition to the yeast strains BY4741 Δbar1, with yeast strains BY4741, BY4742 Δmfα1 Δmfα2, and BY4742 Δbar1 Δmfα1 Δmfα2 that have the aforementioned plasmid combinations.

The functionality of the yeast strain BY4741 p426PGK-EGFP, p423GPD-AR1 has already been proven for different androgenically active substances. The procedure corresponds to the above description for testing the yeast strain BY4741 Δbar1 p426PGK-EGFP, p423GPD-AR1. The following illustration shows in an exemplary fashion. the results for DHT (compare FIG. 7).

A first measurable EGFP signal was observed at 10⁻⁸ mole DHT. Lower androgen concentrations do not differ from the negative control (DMSO). The optimal measuring time is at 26 hours of incubation time because here the highest EGFP signal was detected. In summarizing the above, the EGFP expression in the yeast strain BY4741 p426PGK-EGFP, p423GPD-AR1 can be induced by DHT and thus the androgen receptor is also functionally present in these yeast cells. Further (not illustrated here) measurements show that in addition to DHT also other androgenically active substances effect EGFP formation.

### EXAMPLE 9

### Shmoo Effect

The recognition of the α-factor (yeast pheromone) by surface receptors effects inter alia a directed growth (shmoo effect) of the yeast cells toward the source of the pheromone. Under natural conditions this serves for subsequent fusion with the yeast cell of the opposite mating type.

For detection of androgenically active substances by means of shmoo phenotype two different experimental approaches can be used. The detection of androgenicity of a substance can be realized by a single cell system or by means of an amplification system.

For utilizing the single cell system first an overnight culture of the yeast strain BY4741 MATa p423GPD-AR1, p426PGK-MFα1 in W₀ minimal medium with addition of leucine and methionine is prepared. Incubation is done at 30° C. The yeast strains BY4741 MATa Δbar1 p423GPD-AR1, p416PGK-MFα1; BY4742 MATα Δbar1 Δmfα1 Δmfα2 p423GPD-AR1, p426PGK-MFα1 and BY4742 MATa Δmfα1 Δmfα2 p423GPD-AR1, p426PGK-MFα1 can also be used. In an exemplary fashion, the experimental design by using the yeast strain BY4741 MATa p423GPD-AR1, p426PGK-MFα1 will be explained.

From the overnight culture of the yeast strain BY4741 MATa p423GPD-AR1, p426PGK-MFα1, fresh W₀ minimal medium with addition of leucine and methionine is now inoculated. To the positive control purified α-factor is added. Directed growth of the yeast cells is observed because the α-factor can be recognized by the surface receptors of the a-yeast strain. Based on this control, the androgen-independent induction of the yeast cells can be checked.

As negative control DMSO (dimethyl sulfoxide) or the solvent of the androgenically active substance is added to the medium. In the absence of an androgenically active substance no receptor ligand complex can form and, therefore, the expression of the MFα1 gene by binding to the androgen-responsive elements of the PGK promoter cannot be effected. Since no α-factor can be formed, no shmoo phenotype is produced. Thus, an androgen-independent induction of the MFα1 expression can be excluded.

For analysis of the androgenicity of a testing substance the yeast cells are incubated with different concentrations of this test substance. The incubation of all batches is done at 30° C. The evaluation of the experiment is realized by means of a light microscope. Every hour, cells from each batch are removed and counted by means of a Neubauer counting chamber. In this connection, the total cell number and the number of yeast cells with shmoo phenotype are determined.

For utilizing the amplification system, overnight cultures of all yeast strains to be employed are prepared, as described above. In an exemplary fashion, the experimental design with the following yeast strains BY4741 MATa p423GPD-AR1, p426PGK-MFα1; BY4741 MATa p423GPD-AR1, p426PGK; BY4741 MATa p426PGK-MFα1, p423GPD; BY4741 MATa p426FIG1-MFα1, p423GPD and BY4741 MATa p426FIG1, p423GPD will be described. Performing the method is also possible with the yeast strains BY4741 MATa Δbar1; BY4742 MATa Δmfα1 Δmfα2 and BY4742 MATa Δbar1 Δmfα1 Δmfα2 that carry the aforementioned plasmid combinations. Also the plasmid p416PGK-MFα1 can be used.

For the amplification system, the W₀ minimal medium with addition of leucine and methionine is also inoculated with the overnight cultures.

As positive control, the yeast strains BY4741 MATa p423GPD-AR1, p426PGK-MFα1 (sensor strain) and BY4741 MATa p426FIG1, p423GPD (reporter strain) are co-cultured with different concentrations of an androgen (preferably DHT). The androgen forms with the constitutively formed androgen receptor Isoform1 (AR1) a receptor ligand complex that induces the expression of the MFα1 gene in the sensor strain. The α-factor is secreted and effects a shmoo phenotype of the yeast cells. An amplification effect is not observed because behind the pheromone-responsive FIG1 promoter of the reporter strain there is no MFα1 gene.

As a negative control the common culturing of the yeast strains BY4741 MATa p423GPD-AR1, p426PGK (sensor strain) and BY4741 MATa p426FIG1-MFα1, p423GPD (reporter strain) is carried out in the presence of different androgen concentrations (preferably DHT). Even though it is possible that a receptor ligand complex is formed, the latter however does not induce α-factor production because the MFα1 gene in the sensor strain is missing. Therefore, the expression of the MFα1 gene in the reporter strain cannot be induced. Based on this control, an α-factor-independent formation of the shmoo phenotype can be excluded.

In order to check for the existence of the shmoo phenotype independent of the interaction between androgen receptor and androgen, the yeast strains BY4741 MATa p426PGK-MFα1, p423GPD (sensor strain) and BY4741 MATa p426FIG1-MFα1, p423GPD (reporter strain) are cultured together. In this batch there is no androgen receptor. Thus, the expression of the MFα1 gene can neither be induced in the sensor strain nor in the reporter strain. For the amplification system the yeast strains BY4741 MATa p423GPD-AR1, p426PGK-MFα1 and BY4741 MATa p426FIG1-MFα1, p423GPD are incubated together in the presence of different concentrations of an androgenically active substance.

The evaluation of the experiment is done as described above. Culturing of the test strains in the single cell system as well as in the amplification system based on two different cell types in the presence of DHT caused the formation of the shmoo phenotype that can be observed by light microscope.

### NON-PATENT LITERATURE CITED IN THE DESCRIPTION

Ballensiefen W. and Schmitt H. D. (1997) Periplasmic Bart protease of Saccharomyces cerevisiae is active before reaching its extracellular destination. Eur. J. Biochem. 247: 142-147
Barkai N., Rose M. D. and Wingreen N. S. (1998) Protease helps yeast find mating partners. Nature 396: 422-423.
Sieve G., Lezzi C., Mita G., Rampino P., Perotta C., Villanova L. and Grieco F. (2008) Molecular cloning and heterologous expression of a laccase gene from Pleurotus elyngii in free and immobilized Saccharomyces cerevisiae cells. Appl. Microbiol. Biotechnol. 79: 731-741.
Bovee, T. F. H., Lommerse J. P., Peijnenburg A. A., Fernandes E. A. and Nielen M. W. (2008) A new highly androgen specific yeast biosensor, enabling optimisation of (Q)SAR model approaches. J. Steroid. Biochem. Mol. Biol. 108: 121-131.
Chan R. K. and Otte C. A. (1982) Physiological characterization of Saccharomyces cerevisiae mutants supersensitive to G1 arrest by a factor and a factor pheromones. Mol. Cell. Biol. 2: 21-29.
Chen, J. Ding M. and Pederson D. S. (1994) Binding of TFIID to the yeast CYC1 TATA boxes in yeast occurs independently of upstream activating sequences. Proc. Natl. Acad. Sci. USA 91: 11909-11913.
Chernaik M. L. and Huang P. C. (1991) Differential effect of cysteine-to-serine substitutions in metallothionein on cadmium resistance. Proc. Natl. Acad. Sci. USA 88: 3024-3028.
Chow C. M., Yagüe E., Raguz S., Wood D. A. and Thurston C. F. (1994) The cel3 gene of Agaricus bisporus codes for a modular cellulase and is transcriptionally regulated by the carbon source. Appl. Environ. Microbiol. 60: 2779-2785.
Doesburg P., Kuil C. W., Berrevoets C. A., Steketee K., Faber P. W., Mulder E., Brinkmann A. O. and Trapman J. (1997) Functional in vivo interaction between the amino-terminal, transactivation domain and the ligand binding domain of the androgen receptor. Biochemistry 36: 1052-1064.
Dolan J. W, Kirkman C. and Fields S. (1989) The yeast STE12 protein binds to the DNA sequence mediating pheromone induction. Proc. Natl. Acad. Sci. USA 86: 5703-5707.
Dolan J. W. and Fields S. (1990) Overproduction of the yeast STE12 protein leads to constitutive transcriptional induction. Genes Dev. 4: 492-502.
Dubbink H. J., Hersmus R., Verma C. S., van der Korput H. A., Berrevoets C. A., van Tol J., Ziel-van der Made A. C., Brinkmann A. O., Pike A. C. and Trapman J. (2004) Distinct recognition modes of FXXLF and LXXLL motifs by the androgen receptor. Mol. Endocrinol. 18: 2132-2150.
Ecker D. J., Butt T. R., Sternberg E. J., Neeper M. P., Debouck C., Gorman J. A. and Crooke S. T. (1986) Yeast metallothionein function in metal ion detoxification. J. Biol. Chem. 261: 16895-16900.
Erdman, S., Lin, L., Malczynski, M. and Snyder, M. (1998) Pheromone-regulated genes required for yeast mating differentiation. J. Cell Biol. 140: 461-483.
Fairman C., Zhou X.-L. and Kung C. (1999) Potassium uptake through the TOK1 K⁺ channel in the budding yeast. J. Membr. Biol. 168: 149-157.
Fine T., Leskinen P., lsobe T., Shiraishi H., Morita M., Marks R. S. and Virta M. (2006) Luminescent yeast cells entrapped in hydrogels for estrogenic endocrine disrupting chemical detection. Biosens. Bioelectron. 21: 2263-2269.
Hagen D. C., McCaffrey G., Sprague G. F. Jr. (1991) Pheromone response elements are necessary and sufficient for basal and pheromone-induced transcription of the FUS1 gene of Saccharomyces cerevisiae. Mol. Cell. Biol. 11: 2952-2961.
Harbison C. T., Gordon D. B., Lee T. I., Rinaldi N. J., Macisaac K. D., Danford T. W., Hannett N. M., Tagne J. B., Reynolds D. B., Yoo J., Jennings E. G., Zeitlinger J., Pokholok D. K., Kellis M., Rolfe P. A., Takusagawa K. T., Lander E. S., Gifford D. K., Fraenkel E. and Young R. A. (2004) Transcriptional regulatory code of a eukaryotic genome. Nature 431: 99-104.
Hentges P., Van Driessche B., Tafforeau L., Vandenhaute J., and Carr A. M. (2005) Three novel antibiotic marker cassettes for gene disruption and marker switching in Schizosaccharomyces pombe. Yeast 22: 1013-1019.
Hill J. E., Myers A. M., Koerner T. J. and Tzagoloff A. (1986) Yeast/E. coli shuttle vectors with multiple unique restriction sites. Yeast 2: 163-167.
Hottiger T., Fürst P., Pohlig G. and Heim J. (1994) Physiological characterization of the yeast metallothionein (CUPS) promoter, and consequences of overexpressing its transcriptional activator, ACEI. Yeast 10: 283-296.
Hwang-Shum J. J., Hagen D. C., Jarvis E. E., Westby C. A. and Sprague G. F. Jr. (1991). Relative contributions of MCMI and STE12 to transcriptional activation of a- and α-specific genes from Saccharomyces cerevisiae. Mol. Gen. Genet. 227: 197-204.
Jackson C. L., Konopka J. B. and Hartwell L. H. (1991) S. cerevisiae a pheromone receptors activate a novel signal transduction pathway for mating partner discrimination. Cell 67: 389-402.
Janes M., Meyhack B., Zimmermann W. and Hinnen A. (1990) The influence of GAP promoter variants on hirudin production, average copy number and cell growth in Saccharomyces cerevisiae. Curr. Genet. 18: 97-103.
Jenster G., van der Korput H. A., Trapman J. and Brinkmann A. O. (1995) Identification of two transcription activation units in the N-terminal domain of the human androgen receptor. J. Biol. Chem. 270: 7341-7346.
Kaiser C., Michaelis S. and Mitchell A. (1994) Methods in Yeast Genetics. New York, Cold Spring Harbor Laboratory Press.
Kronstad J. W., Holly J. A. and MacKay V. L. (1987) A yeast operator overlaps an upstream activation site. Cell 50: 369-377.
Langley E., Zhou Z. X. and Wilson E. M. (1995) Evidence for an anti-parallel orientation of the ligand-activated human androgen receptor dimer. J. Biol. Chem. 270: 29983-29990.
Leberer E., Thomas D. Y. and Whiteway M. (1997) Pheromone signaling and polarized morphogenesis in yeast. Curr. Opin. Genet. Dev. 7: 59-66.
Mackay V. and Manney T. R. (1974) Mutations affecting sexual conjugation and related processes in Saccharomyces cerevisiae. I. Isolation and phenotypic characterization of nonmating mutants. Genetics 76: 255-271.
Marsh, L. and Rose, M. D. (1997) The pathway of cell and nuclear fusion during mating in S. cerevisiae. In: Pringle, J. R., Broach, J. R. and Jones, E. W. (eds.) The molecular and cellular biology of the yeast Saccharomyces. Cold Spring Laboratory press, New York.
McNabb D. S., Reed R. and Marciniak R. A. (2005) Dual luciferase assay system for rapid assessment of gene expression in Saccharomyces cerevisiae. Eukaryot. Cell 4: 1539-1549.
Mille Y., Girard J.-P., Beney L. and Gervais P. (2005) Air drying optimization of Saccharomyces cerevisiae through ist water-glycerol dehydration properties. J. Appl. Microbiol. 99: 376-382
Mooradian A. D., Morley J. E. and Korenman S. G. (1987) Biological actions of androgens. Endocr. Rev. 8: 1-28.
Muller, E. M., Mackin, N. A., Erdman, S. and Cunningham, K. W. (2003) Fig1p facilitates Ca²⁺ influx and cell fusion during mating of Saccharomyces cerevisiae. J. Biol. Chem. 278: 38461-38469.
Mumberg D., Mailer R. and Funk M. (1995) Yeast vectors for the controlled expression of heterologous proteins in different genetic backgrounds. Gene 156: 119-122.
Opekarova M., Caspari T., Pinson B., Brethes D. and Tanner W. (1998) Post-translational fate of CAN1 permease of Saccharomyces cerevisiae. Yeast 14: 215-224.
Perez-Castineira J. R., Lopez-Marques R. L., Villalba J. M., Losada M. and Serrano A. (2002) Functional complementation of yeast cytosolic pyrophosphatase by bacterial and plant H⁺-translocating pyrophosphatases. Proc. Natl. Acad. Sci. USA 99: 15914-15919.
Protchenko O., Shakoury-Elizeh M., Keane P., Storey J., Androphy R. and Philpott C. C. (2008) Role of PUG1 in inducible porphyrin and heme transport in Saccharomyces cerevisiae. Eukaryot. Cell 7: 859-871.
Purvis I. J., Chotai D., Dykes C. W., Lubahn D. B., French F. S., Wilson E. M. and Hobden A. N. (1991) An androgen-inducible expression system for Saccharomyces cerevisiae. Gene 106: 35-42.
Roberts C. J., Nelson B., Marton M. J., Szoughton R., Meyer M. R., Bennett H. A., He Y. D., Dai H., Walker W. L., Hughes T. R., Tyers M., Boone C. and Friend S. H. (2000) Signaling and circuitry of multiple MAPK pathways revealed by a matrix of global gene expression profiles. Science 287: 873-880.
Routledge E. J. and Sumpter J. P. (1996) Estrogenic activity of surfactants and some of their degradation products assessed using a recombinant yeast screen. Environ. Toxicol. Chem. 15: 241-248.
Roy A. K., Lavrovsky Y., Song C. S., Chen S., Jung M. H., Velu N. K., Bi B. Y. and Chatterjee B. (1999) Regulation of androgen action. Vitam. Horm. 55: 309-352.
Segall J. E. (1993) Polarization of yeast cells in spatial gradients of α-mating factor. Proc. Natl. Acad. Sci. USA 90: 8332-8336.
Sekler I., Kopito R. and Casey J. R. (1995) High level expression, partial purification, and functional reconstitution of the human AE1 anion exchanger in Saccharomyces cerevisiae. J. Biol. Chem. 270: 21028-21034.
Sohoni P. and Sumpter J. P. (1998) Several environmental oestrogens are also anti-androgens. J. Endocrinol. 158: 327-339.
Sprague G. F. Jr and Herskowitz I. (1981) Control of yeast cell type by the mating type locus. I. Identification and control of expression of the a-specific gene BAR1. J. Mol. Biol. 153: 305-321.
Tkacz J. S. and MacKay V. L. (1979) Sexual conjugation in yeast: cell surface changes in response to the action of mating hormones. J. Cell Biol. 80: 326-333.
Wilson C. M. and McPhaul M. J. (1994) A and B forms of the androgen receptor are present in human genital skin fibroblasts. Proc. Natl. Acad. Sci. USA 91: 1234-1238.
Zhang, N.-N., Dudgeon D. D., Paliwal S., Levchenko A., Grote E. and Cunningham K. W. (2006) Multiple signaling pathways regulate yeast cell death during response to mating pheromones. Mol. Biol. Cell 17: 3409-3422.

What is claimed is:

1.-26. (canceled)

27. A method for verifying and/or identifying hormonally effective substances, comprising the steps of:
a) to a sample that comprises presumably a hormonally active substance a yeast culturing medium or buffer is added,
b) the sample to which the yeast culturing medium or buffer has been added is contacted with haploid yeast cells that comprise the following features 1 to 3, wherein either all features 1 to 3 are realized by a single type of haploid yeast cells or the features 1 and 2 are realized by a first type of haploid yeast cells and the feature 3 by a second type of haploid yeast cells:
1. the DNA sequence that codes for a heterologous hormone receptor is subjected to the control of a constitutive promoter and is functionally expressed;
2. the DNA sequence coding for a yeast pheromone is subjected to the control of a promoter that can be regulated by the heterologous hormone receptor;
3. a gene that codes for a receptor for the yeast pheromone is constitutively and functionally expressed,
wherein the activation of the heterologous hormone receptor by the hormonally active substance causes the expression of the DNA sequence coding for the yeast pheromone and the secretion of the pheromone from the haploid yeast cell; and
c) a morphological change and/or a physiological change of the haploid yeast cells are detected.

28. The method according to claim 27, wherein the morphological change of the haploid yeast cells is a directed cell wall growth caused by the action of the yeast pheromone.

29. The method according to claim 27, wherein the morphological change of the haploid yeast cells is detected by a light microscope.

30. The method according to claim 27, wherein the morphological change of the haploid yeast cells is detected by a physical measurement of turbidity.

31. The method according to claim 27, wherein the morphological change of the haploid yeast cells is a cell cycle arrest caused by the action of the yeast pheromone.

32. The method according to claim 27, wherein the physiological change of the haploid yeast cells comprises the expression of a specific gene.

33. The method according to claim 32, wherein the specific gene codes for a marker protein.

34. The method according to claim 32, wherein the specific gene codes for a fluorescent protein.

35. The method according to claim 32, wherein the specific gene codes for a protein that fluoresces green (GFP), yellow (YFP), blue (BFP), cyan (CFP), or red (dsRed).

36. The method according to claim 27, wherein the hormone receptor is an intracellular hormone receptor.

37. The method according to claim 27, wherein the hormone receptor is a nuclear hormone receptor.

38. The method according to claim 27, wherein the hormone receptor is a steroid receptor.

39. The method according to claim 27, wherein the hormone receptor is selected from androgen receptors and estrogen receptors.

40. The method according to claim 27, wherein the constitutive promoter is selected from the group consisting of ADH1, GPD, TEF2, CYC, PGK1, PGK1, SPT15, PMA1, SUF14, GAPFL, TPI1 and TDH3 promoters.

41. The method according to claim 27 for detecting hormonally active substances.

42. The method according to claim 41 for detecting hormonally active substances in urine samples, blood samples, and serum samples.

43. The method according to 42 for detecting doping agents in urine samples.

44. The method according to claim 27 for evaluating loading of environmental samples with hormonally active substances.

45. The method according to claim 44 wherein the environmental samples are water samples or tissue samples.

46. The method according to claim 27 for evaluating hormonal activity of synthetic or natural substances.

47. The method according to claim 27 for screening substance libraries or complex substance mixtures.

48. The method according to claim 27 for identifying potentially hormonally active substances for pharmaceutical application.

49. The method according to claim 27 for toxicological screening of waste water, drinking water, packaging materials, food stuffs or food supplements with regard to undesirable hormonal ingredients.

50. The method according to claim 27 for screening with respect to use of hormones in fattening of animals.

51. Haploid yeast cells with the following features:
a DNA sequence that codes for a heterologous hormone receptor is subjected to the control of a constitutive promoter and is functionally expressed;
a DNA sequence that codes for a yeast pheromone is subjected to the control of a promoter that can be regulated by the heterologous hormone receptor;
wherein the activation of the heterologous hormone receptor by the hormonally active substance causes the expression of the DNA sequence coding for the yeast pheromone and the secretion of the yeast pheromone from the haploid yeast cell.

52. The haploid yeast cells according to claim 51, constitutively and functionally expressing a gene that codes for a receptor of the yeast pheromone.

53. The haploid yeast cells according to claim 51 for detecting hormonally active substances.

54. The haploid yeast cells according to claim 53 for detecting hormonally active substances in urine samples, blood samples, and serum samples.

55. The haploid yeast cells according to 53 for detecting doping agents in urine samples.

56. The haploid yeast cells according to claim 51 for evaluating loading of environmental samples with hormonally active substances.

57. The haploid yeast cells according to claim 56 wherein the environmental samples are water samples or tissue samples.

58. The haploid yeast cells according to claim 51 for evaluating hormonal activity of synthetic or natural substances.

59. The haploid yeast cells according to claim 51 for screening substance libraries or complex substance mixtures.

60. The haploid yeast cells according to claim 51 for identifying potentially hormonally active substances for pharmaceutical application.

61. The haploid yeast cells according to claim 51 for toxicological screening of waste water, drinking water, packaging materials, food stuffs or food supplements with regard to undesirable hormonal ingredients.

62. The haploid yeast cells according to claim 51 for screening with respect to use of hormones in fattening of animals.
